(19) United States
(12) Patent Application Publication
(10) Pub. No.: US 2025/0153170 A1
(43) Pub. Date: May 15, 2025

(54) FLUID CONTROL ADAPTERS

(71) Applicant: Carterra, Inc., Salt Lake City, UT (US)

(72) Inventors: Viatcheslav PETROPAVLOVSKIKH, Louisville, CO (US); James SMITH, Bountiful, UT (US); Bruce GALE, Taylorsville, UT (US); Andrew HATCH, West Jordan, UT (US); James TRAUBA, Salt Lake City, UT (US)

(21) Appl. No.: 18/947,269

(22) Filed: Nov. 14, 2024

Related U.S. Application Data
(60) Provisional application No. 63/598,620, filed on Nov. 14, 2023.

(51) Int. Cl. B01L 3/00

(52) U.S. Cl. CPC B01L 3/502746; B01L 3/502715; B01L 2200/028; B01L 2200/16; B01L 2300/0663; B01L 2300/123; B01L 2400/084

(57) ABSTRACT

A fluid control adapter can include a proximal adapter surface including an array of proximal adapter openings to receive fluid from a fluid directing body when fluidly connected therewith, and a distal adapter surface including an array of distal adapter openings to pass fluid to an applicator tip when fluidly connected therewith. An adapter volume can define a plurality of adapter microfluidic channels individually fluidically connecting one or more proximal adapter openings with one or more distal adapter openings. The fluid control adapter can also include a fluid modification architecture within the adapter volume to interact with fluid contained within or passing through one or more of the adapter microfluidic channels.

The present application claims the benefit of U.S. Provisional Patent Application No. 63/598,620, filed on Nov. 14, 2023, the entirety of which is incorporated herein by reference.

### BACKGROUND

Biomolecular interaction sensing and imaging systems are used by researchers in the academic, pharmaceutical, and biotechnology sectors to observe, evaluate, and/or characterize binding interactions, e.g., antibody characterization, proteomics, vaccines, immunogenicity, biopharmaceutical development and production, etc. Numerous commercial biosensor instruments based on microarray approaches are available, including label-free biosensor and flow-through cell instruments, as well as other “printing” systems, e.g., pin printing, piezo printing, microfluidic array printing, etc. In some systems, after applying the material(s) of interest on a substrate (ligand or similar substance), the sample (analyte) is loaded onto the biosensor where a binding interaction between the ligand and the sample occurs which can be further evaluated or characterized, or alternatively, no binding is observed which can be noted. In other systems, the application of substances is done in the instrument and then the binding analyte is delivered to the applied spots using the same flow cell arrays and the binding reaction is observed.

### BRIEF DESCRIPTION OF THE DRAWINGS

FIG. 1 illustrates a schematic view of an example substance spotter system including an example modular microfluidic flow cell array in accordance with the present disclosure;

FIG. 2A illustrates a perspective view of an example modular microfluidic flow cell array including a fluid directing body and a fluid control adapter in accordance with the present disclosure;

FIG. 2B illustrates a top plan view of an alternative example fluid control adapter compared to that shown in FIG. 2A in accordance with the present disclosure;

FIG. 2C illustrates a perspective view of an enlarged portion of the example modular microfluidic flow cell array shown in FIG. 2A in accordance with the present disclosure;

FIG. 2D illustrates a perspective view of an alternative enlarged portion of the example fluid control adapter shown in FIG. 2A utilizing magnetic connection between the fluid directing body and the fluid control adapter in accordance with the present disclosure;

FIG. 3 illustrates a top plan view and a cross-sectional view of an example fluid directing body adapted for fluid flow path combining in accordance with the present disclosure;

FIG. 4 illustrates a top plan view and a cross-sectional view of an example fluid directing body adapted for fluid flow path dividing in accordance with the present disclosure;

FIG. 5 illustrates a perspective view of an example modular fluid control adapter system with multiple stacked fluid control adapters providing multiple functionalities in accordance with the present disclosure;

FIG. 6 illustrates a front plan view of an example fluid directing body and an end plan view plan depicting a distal body surface and distal body openings of the fluid directing body in accordance with the present disclosure;

FIG. 7 illustrates a plan view of a proximal adapter surface of an example first fluid control adapter along with its cross-sectional view, and also illustrates a cross-sectional view of an example second fluid control adapter and application substrate in accordance with the present disclosure;

FIG. 8 illustrates a plan view of proximal tip surfaces of two example application tips along with their cross-sectional views in accordance with the present disclosure;

FIG. 9 illustrates a perspective view of a portion of a modular microfluidic flow cell array having multiple fluid control adapters integrated with or attached to an application surface in accordance with the present disclosure; and

FIG. 10 illustrates a perspective view of an alternative modular microfluidic flow cell array having multiple fluid control adapters integrated with or attached to an application surface in accordance with the present disclosure.

### DETAILED DESCRIPTION

In accordance with examples herein, the present disclosure provides fluid control adapters, modular fluid control adapter systems, modular microfluidic flow cell arrays, and methods of applying substances for analysis to an application surface, among other embodiments. These fluid control adapters, modular fluid control adapter systems, modular microfluidic flow cell arrays, and methods can be used, for example, for high throughput binding reaction observations, evaluations, characterization, etc.

In accordance with examples of the present disclosure, a fluid control adapter can include a proximal adapter surface including an array of proximal adapter openings to receive fluid from a fluid directing body when fluidly connected therewith, and a distal adapter surface including an array of distal adapter openings to pass fluid to an applicator tip when fluidly connected therewith. An adapter volume can define a plurality of adapter microfluidic channels individually fluidically connecting one or more proximal adapter openings with one or more distal adapter openings. The fluid control adapter can also include a fluid modification architecture within the adapter volume to interact with fluid contained within or passing through one or more of the adapter microfluidic channels.

In another example, a modular fluid control adapter system can include a first fluid control adapter and a second fluid control adapter. The first fluid control adapter can include a first adapter microfluidic channel fluidically connecting one or more proximal adapter openings defined by a proximal adapter surface with one or more distal adapter openings defined by a first distal adapter surface. The second fluid control adapter can include a second adapter microfluidic channel fluidically connecting one or more proximal adapter openings defined by a second proximal adapter surface with one or more distal adapter openings defined by a second distal adapter surface. The first distal adapter surface can be releasably sealable against the second proximal adapter surface to allow the flow of fluid from within the first adapter microfluidic channel to within the second adapter microfluidic channel. In further detail, the first adapter microfluidic channel can interact mechanically, optically, chemically, biologically, or thermally with fluid when introduced therein in a manner that is different than how the second adapter interacts with the fluid.

In another example, a modular microfluidic flow cell array can include a fluid directing body defining a plurality of body microfluidic channels fluidically connecting source fluid openings with distal body openings at a distal body surface, and an applicator tip. The applicator tip can include a proximal tip surface defining proximal tip openings and a distal tip surface defining distal tip openings fluidically connected to the proximal tip openings. The distal tip openings can be arranged to provide fluid communication between the applicator tip and an application surface when the distal tip surface is contacted therewith. The modular microfluidic flow cell array can also include a tip coupling feature at or adjacent to the proximal tip surface, wherein the tip coupling feature is removably connectable to the fluid directing body to provide fluidic communication between the distal body openings and the proximal tip openings.

In another example, a modular microfluidic flow cell array can include a fluid directing body defining a plurality of body microfluidic channels fluidically connecting source fluid openings with distal body openings at a distal body surface, a first fluid control adapter, and an applicator tip. The first fluid control adapter can include a first adapter microfluidic channel fluidically connecting one or more proximal adapter openings defined by a proximal adapter surface with one or more distal adapter openings defined by a first distal adapter surface. The proximal adapter surface can be removably connectable with the distal body surface of the fluid directing body. The applicator tip can be removably connectable with the first distal adapter surface of the first fluid control adapter. When the fluid directing body, the first fluid control adapter, and the applicator tip are fluidically connected, substance applied to an application surface occurs as fluid passes from the fluid directing body, through the first fluid control adapter, and through the applicator tip depositing the substance to the application surface. For example, the applicator tip can provide for depositing the substance on the application surface via a flow chamber in some examples.

In another example, a method of treating an application surface for analysis of a substance can include docking a first modular microfluidic flow cell array on an application surface and flowing a first fluid through the first modular microfluidic flow cell array including through the first fluid control adapter. In this example, the first modular microfluidic flow cell array can include a fluid directing body fluidly connected to a first fluid control adapter. The method can further include undocking the first modular microfluidic flow cell array from the application surface, and removing the first fluid control adapter from the fluid directing body. In further detail, the method can include fluidly connecting a second fluid control adapter to the fluid directing body to form a second modular microfluidic flow cell array, docking the second modular microfluidic flow cell array on the application surface, and flowing a second fluid through the second modular microfluidic flow cell array including through the second fluid control adapter.

In another example, a method of applying substances for analysis to an application surface can include fluidly connecting a fluid directing body with a first applicator tip to form a first microfluidic flow cell array, docking the applicator tip of the first modular microfluidic flow cell array on an application surface, flowing a first fluid through the fluid directing body and the first applicator tip, and disconnecting the fluid directing body from the first applicator tip. In further detail, the method can include fluidly connecting the fluid directing body with a second applicator tip to form a second microfluidic flow cell array, docking the second applicator tip of the second modular microfluidic flow cell array on the application surface, and flowing a second fluid through the fluid directing body and the second applicator tip. In this example, flowing the first fluid and flowing the second fluid results in application of at least one set of substance spots on the application surface.

It is noted that when discussing the fluid control adapters, modular fluid control adapter systems, modular microfluidic flow cell arrays, and methods of applying substances for analysis to an application surface, these discussions are considered applicable to other examples whether or not they are explicitly discussed in the context of that example unless expressly indicated otherwise. Thus, for example, when discussing a certain type of fluid or deposition substance, or material of construction, or the like in the context of the fluid control adapters, such disclosure is also relevant to and directly supported in context of the other example modular fluid control adapter systems, modular microfluidic flow cell arrays, methods of applying substances for analysis to an application surface, and vice versa. Furthermore, for simplicity and illustrative purposes, numerous specific details are set forth in order to provide a thorough understanding of the present disclosure. It will be readily apparent however, that the present disclosure can be practiced without limitation to some of these specific details. In other instances, certain methods, systems, materials, and structures have not been described in detail so as not to obscure the present disclosure.

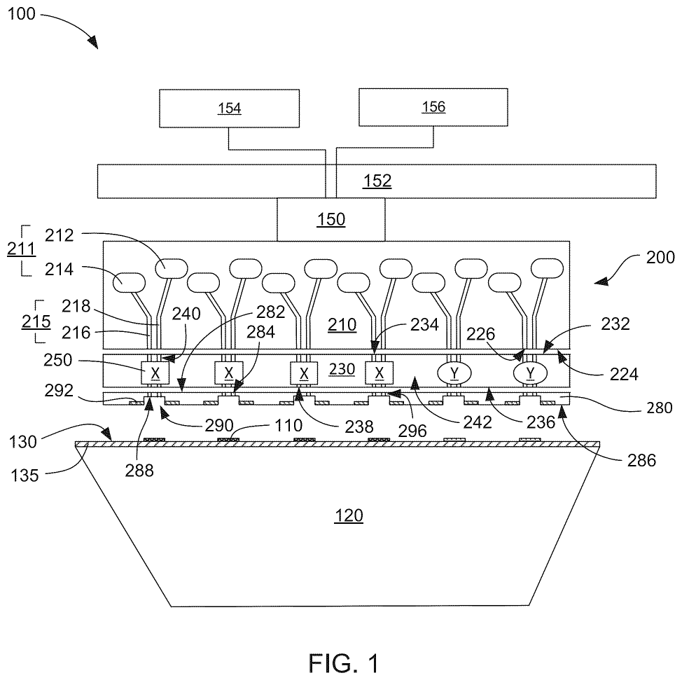

Referring now to FIG. 1, an example substance spotter system 100 is shown that includes a modular microfluidic flow cell array 200 of the present disclosure. As an initial matter, the modular microfluidic flow cell array is shown as being present in the context of an example substance spotter system, including a variety of positioning and force sensors, e.g., z-axis positioner 150, xy-axis positioner 152, positioning sensor 154, force sensor 156, etc. In some examples, there may also be a tip positioning sensor 292 positioned as part of the modular microfluidic flow cell array, which as shown in this example, is present at a distal tip surface 286 of an applicator tip 280. The substance spotter system shown in this specific embodiment also depicts solid optical material 120, which in this example is an optical prism with an application surface 130 provided by a sensor chip 135. This arrangement may be particularly useful for surface plasmon resonance (SPR) applications. However, it is noted that other arrangements can be used with the modular microfluidic flow cell arrays of the present disclosure, including the use of other positioners and/or other application surfaces. This example is merely to provide one example context to which these arrays can be used.

Referring now more specifically to the modular microfluidic flow cell array 200 shown by way of example in FIG. 1, the array can include a fluid directing body 210, a fluid control adapter 230, and an adapter tip 280. Notably, for clarity these three structures are shown as not being connected so that adjoining surfaces and various channel openings can be shown. However, it is noted that in operation, the gap shown between these three structures relative to one another would typically be closed so that the various channels would be fluidically connected. Regarding the fluid directing body, this structure can define a plurality of body microfluidic channels 215. The plurality of body microfluidic channels can include multiple pairs of a first body microfluidic channel 216 and a second body microfluidic channel 218, for example. It is noted that though the FIGS. hereinafter typically show pairs of body microfluidic channels, in some examples, there may be instances where there is no return fluid flow. The body microfluidic channels in this example fluidically connect source fluid openings 211 with distal body openings 226 located at a distal body surface 224. The “distal body surface” can be an interface surface of the fluid directing body that interfaces with a fluid control adapter to connect body microfluidic channels together to form or contribute to the formation of flow cells. The body microfluidic channels of the fluid directing body can be directly connected to matching adapter microfluidic channels, or these microchannels can be matched differently. For example, one or more of the proximal adapter openings of a fluid control adapter can be large enough to span distal body openings of the fluid directing body. This can provide fluid combining, for example. In this example, the fluid control adapter may be considered to be a first fluid control adapter and an applicator tip 280 can be attached thereto. The applicator tip can be considered to be a second fluid control adapter in some examples. The applicator tip provides the function of application of a substance from a fluid flowing therethrough via its flow chambers 290, thus leaving substance spots 110 behind on an application surface 130, which in this instance is the application surface described previously that includes a sensor chip 135 deposited on a solid optical material 120, e.g., optical prism. In some examples, the applicator tip may have other functionalities in addition to substance deposition, such as any of those described herein that relate to the various fluid control adapters.

In further detail regarding the fluid control adapter 230, this structure can include adapter microfluidic channels 240, e.g., multiple pairs of a first adapter microfluidic channel and a second adapter microfluidic channel. The adapter microfluidic channels are defined within an adapter volume of material 242. The adapter microfluidic channels fluidically connect one or more proximal adapter openings 234 defined by a proximal adapter surface 232 with one or more distal adapter openings 238 defined by a distal adapter surface 236. In accordance with the present disclosure, the term “proximal” refers to a surface or interface openings that are further from the application surface 130 with respect to fluid flow. Thus, the term “distal” refers to a surface or interface openings that are closer to the application surface with respect to fluid flow. These terms can be used to describe surfaces and openings of any structure of the modular microfluidic flow cell arrays described herein, including surfaces of the fluid directing bodies, the fluid control adapters, the applicator tips, etc.

In some examples, the fluid control adapter 230 can have an adapter volume that is softer at the proximal adapter surface than at the distal adapter surface. In other examples, the adapter volume can be harder at an interior location thereof compared to the proximal adapter surface, the distal adapter surface, or both. In yet other examples, in addition to the plurality of adapter microfluidic channels that pass through the fluid control adapter, the adapter can be configured so that one or more of the adapter microfluidic channels are blocking channels. For example, some of the channels could be blocking channels, or in some instances, all of the channels may be blocking channels, such as may be useful for fluid priming. Example materials that can be used for the fluid control adapter may include various rigid or soft materials, such as plastic, elastomer, composites, ceramics, adhesives, gels, silicon, glass, metal, material suitable for a printed circuit board, materials suitable for three-dimensional printing, molding, laminating, diffusion bonding, or the like.

In addition to the fluid control adapter 230 being removably connectable with the fluid directing body 210, the fluid control adapter can also be removably connectable with an applicator tip 280. For example, the distal adapter surface 236 can also be removably connectable with an applicator tip 280 along its proximal tip surface 282, which defines multiple proximal tip openings 284, which in example are positioned within a flow chamber 290 to provide fluid to and allow removal of fluid therefrom. Fluidically connecting the fluid directing body, the fluid control adapter, and the applicator tip typically provides a plurality of fluid flow paths from the fluid directing body, through the fluid control adapter, and through the applicator tip to generate a substance spot 110 on an application surface 130 using the flow chamber defined by the distal surface of the applicator tip. The fluid is introduced into or flowed through the flow chamber in this example via tip microfluidic channels 296 that pass fluid between the proximal tip openings and distal tip openings 288 defined by a distal tip surface 286. For example, substance applied to an application surface can occur as fluid passes from the fluid directing body, through the first fluid control adapter, and through the applicator tip depositing the substance to the application surface. In some examples, there may be a return flow path returning fluid from the flow chamber through the tip microfluidic channels, the adapter microfluidic channels, and into or through one or both of the pair of body microfluidic channels.

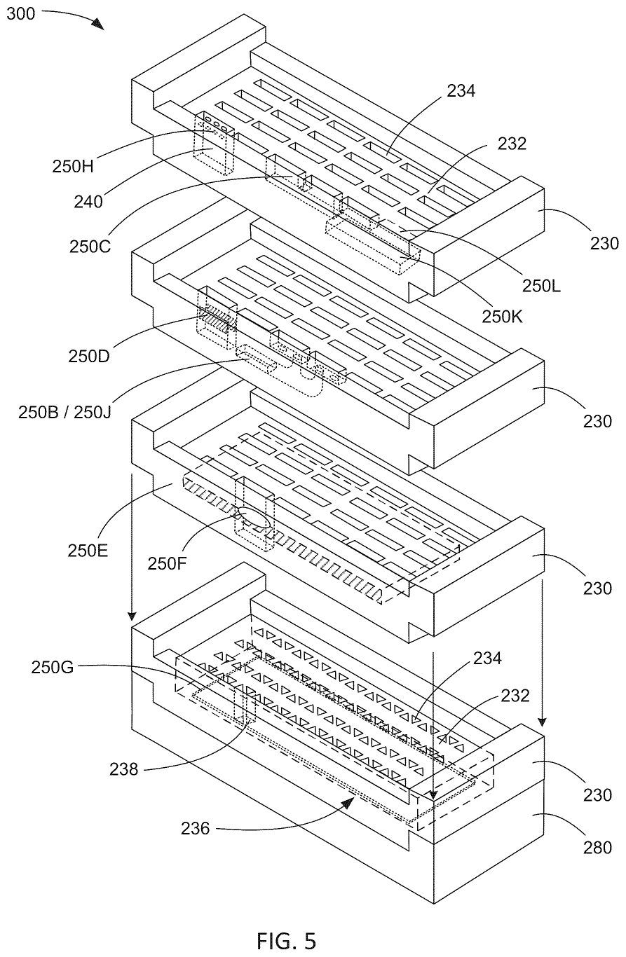

The fluid control adapter 230 can include one or more fluid modification architecture or component 250. In the example shown at FIG. 1, the fluid control adapter is shown with two different fluid modification architectures or components positioned in parallel with one another at “X” and “Y.” However, it is noted that a single fluid modification architecture or component type may be present, or multiple fluid modification architectures or components can be arranged within a fluid control adapter in series along a common flow path. Thus, a fluid control adapter may include a single fluid modification architecture or component, or may include a first and a second (third, forth, etc.) fluid modification architecture in parallel or in series relative to the flow path(s) with the adapter microfluidic channels 240 within the adapter volume of material 242. For example, a fluid control adapter may include a dry reagent and a heater component to both introduce reagent to the passing fluid and also heat the fluid, such as for nucleic acid amplification. In another example, a fluid control adapter may be configured to both combine and mix fluids. These functions can be carried out in a single fluid control adapter, or in a more flexible system, can be carried out using multiple fluid control adapters that are configured to be joined together (as described in greater detail at FIG. 5). It is noted that these example combinations of fluid modification architectures or components are not intended to be limiting, but to illustrate the flexibility of the fluid control adapters of the present disclosure.

Examples of fluid modification architectures or components include mechanical architectures, such as architectures suitable for fluid dividers, fluid combiners, fluid mixers, fluid resistors, pumps, valves, or a combination thereof; electrical components, such as electrodes or other structures to introduce electrical current, electrophoresis, electromagnetic frequency (to move or sense fluids), etc., capacitors, resistors, inductors, piezoelectric components, antennas, heaters, coolers, or a combination thereof; valves, such as mechanical valves, microelectromechanical systems (MEMS) valves, bubble valves, wax valves, rheologic valves, or a combination thereof; sensors, such as optical sensors, chemical sensors, MEMS sensors, electrical sensors, biological sensors, capacitance sensors, pressure sensors, accelerometers, temperature sensors, or a combination thereof; thermocontrollers, such as heaters, coolers, heat sinks, phase change heat pipes, ducts, absorbers, or a combination thereof; reagent such as dry reagent or liquid reagent; particle or cellular capture or sorting architecture; or a combination thereof.

In addition to that shown by way of example in FIG. 1, any number of devices may be attached to the substance spotter system 100. A few examples may include pumps, blowers, vacuums, fluid lines, heating/cooling jackets, mounting hardware, and reservoirs such as beakers or microtiter plates. The various flow cells formed by connecting the components of the modular microfluidic flow cell array 200 may both direct fluid and return fluid from the application surface 130 to different reservoirs or to the same reservoir connected to the source fluid openings 211 of the fluid directing body 210. In some instances, by returning fluid to the same reservoir for reapplication or by utilizing bi-directional flow, increased binding of a molecule at a location of a substance spot on the application surface may be possible even with fluids in which the molecule is present in very low concentrations.

In further detail, substances may be moved through the flow cells (once formed by connecting the fluid directing body with any fluid control adapters and an applicator tip) either by pressure-flow, gravity-flow, electrokinetic processes, air pressure, and/or any other suitable method. In one specific example, creating pressure-flow and gravity-flow can be accomplished by using pumps and/or vacuums. When using a pair of body microfluidic channels 215 with a pair of source fluid openings 211, for example, if the pressure at a second source fluid opening 214 is lower than the pressure at a first source fluid opening 212, a siphon may be established for flowing a substance through the flow cell, with fluid contacting the application surface 130 at the flow chamber 290. Similarly, air pressure may be used, for example, to push a plug of a viscous gel along the fluid pathway to propel a solution, or a reservoir may be pressurized to propel the solution. In other examples, charged compounds, such as negatively charged DNA, can benefit from the use of electrokinetic pumps to move such charged substances within the flow cell for application to or interaction with the application surface. Additionally, in some instances, doped or coated interior walls of any or all of the various microfluidic channels can be used to increase or modify a charge, e.g., negative charge, which can act to reduce the friction between negatively charged substances and the interior of the conduits.

The applicator tip 280 may include a distal tip surface that defines both interface portions that contact the application surface as well as the flow chambers 290 that are used to pool fluid and interrogate or apply substance to the application surface. Though the distal tip surface that contacts the application surface is shown as being generally flat, other configurations can be used as well. For example, the distal tip surface can merely be the flow chambers defined by a bundle of microtubules. In this embodiment, if the orifices are circular, the distal tip surface could be in the form of a collection of rings that define individual flow chambers.

Thus, the distal tip surface could include some open gaps between the collection of rings rather than be configured as a solid surface. These open gaps could remain open, or could be filled with other material. For example, microtubules could be held together by an epoxy used to fill in the gaps between the channels, and the cured epoxy and the microtubules defining the flow chambers could then be cut and/or polished to form a smooth surface.

Regardless of the configuration of the applicator tip, the distal tip surface 286 may be pressed against the application surface 130 to form a seal about the flow chambers 290 so that the flow chambers may form a sealed chamber defined by recessed portions of the distal tip surface and the application surface. Typically, a fluid tight seal may be formed to prevent contact or cross-talk between spots when applied to the application surface. By preventing contact or cross-talk between substance spots, the modular microfluidic flow cell arrays 200 described herein can be suitable for applications that would benefit from internal referencing, described in greater detail hereinafter.

The distal tip surface 286 (or spotter face) that interfaces with the application surface 130 can be any size or geometry. Examples include distal tip surfaces designed to cover 76 cm×2 6 cm microscope slides, or any of a number of commercially available wafers, e.g., 25 mm, 50.8 mm, 76.2 mm, 100 mm, 125 mm, 150 mm, 200 mm, 300 mm, etc. Additionally, the distal tip surface can be designed to correspond to any substrate or structure on a substrate. For example, if an application surface includes ridges, the distal tip surface may be modified to have valleys that mate with the substrate ridges or vice versa. The distal tip surface may also be made rigid or be of sufficient flexibility to conform to an application surface. In some embodiments, the distal tip surface may be designed to facilitate integrating the spotter with an analysis platform. For example, the distal tip surface may be sealed effectively on an application surface that can serve as the transducer face of any of a number of analysis platforms, such as a surface plasmon resonance (SPR) or a surface plasmon resonance imaging (SPRi) platform.

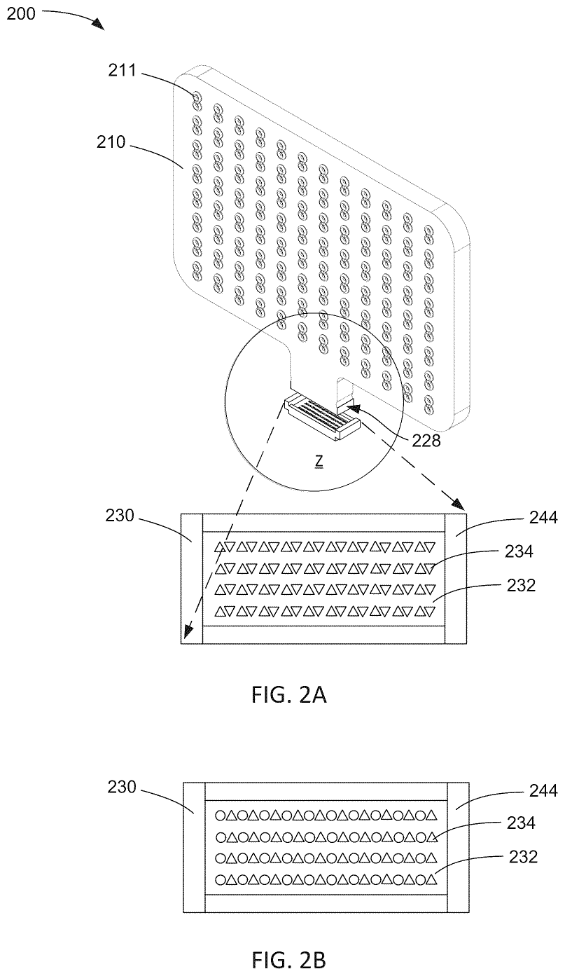

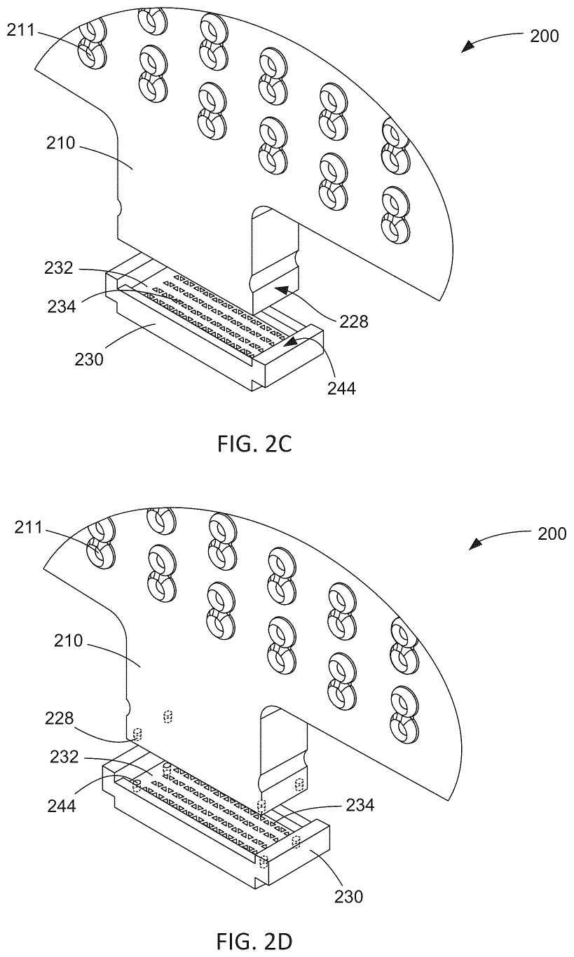

Referring now to FIG. 2A, a perspective view of an example modular microfluidic flow cell array 200 is shown which includes a fluid directing body 210 and a fluid control adapter 230. A top plan view of the fluid control adapter is also shown for additional clarity. Furthermore, a portion of the modular microfluidic flow cell array notated at “Z” is shown in greater detail at FIG. 2C. As shown in these examples, the fluid directing body includes source fluid openings 211 or fluid ports, which are visible in this illustration. The source fluid openings are shown as being grouped in pairs, which can be convenient for visually showing a user which ports connect fluidically to a common flow chamber after passing through the modular microfluidic flow cell array. Notably, as the fluid control adapter has the ability to provide functionalities, such as combining channels, dividing channels, etc., in some instances, fluids can be redirected in ways not typical for unidirectional or bidirectional fluid flow. For example, two adjacent source fluid openings could provide fluid ingress, and two other adjacent source fluid openings could provide fluid egress. In other words, because the fluid control adapter provides flexibility of use with respect to designing fluid flow, after the fluid leaves the fluid directing body, any number of redirections of fluid can occur prior to returning fluid back into the fluid directing body for disposal, analysis, bi-directional flow, reintroduction into the flow cell, etc.

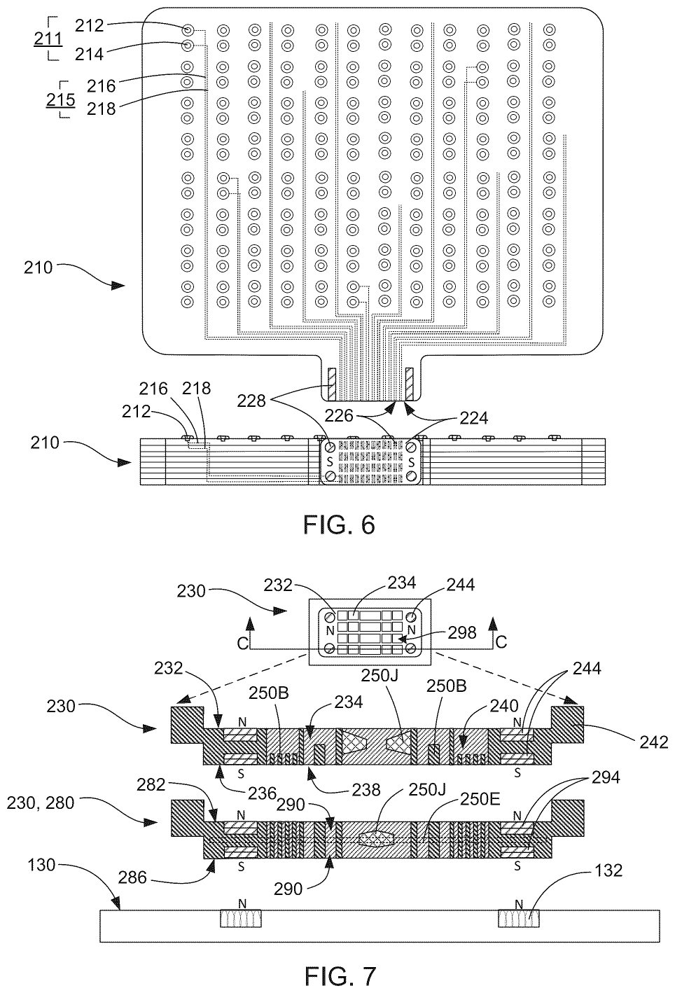

In FIG. 2A and FIG. 2C, the fluid control adapter 230 is shown to include a proximal adapter surface 232 defining a plurality of proximal adapter openings 234, which in this instance are triangle-shaped. FIG. 2B illustrates a similar arrangement, but includes proximal adapter openings having two different shapes, e.g., triangular and circular. The proximal adapter openings are arranged as a 4×20 array of openings. However, the proximal adapter openings and/or the distal adapter openings can be configured in any of a number of configurations, such as in rows and columns (as shown), shaped patterns, e.g., circles, rectangles, triangles, other polygons, etc., staggered patterns, random patterns, etc. As shown in FIGS. 2A-2C, the fluid control adapters 230 have an adapter coupling feature 244, which in these instances, include a pair of laterally positioned flanges that are sized to pressure fit against a body coupling feature 228. For example, the adapter coupling feature may be in the form of rigid flanges and the body coupling feature may be a sufficiently soft material that can be compressed within the rigid flanges. FIG. 2D, on the other hand, includes many of the same features as that shown in FIGS. 2A and 2C, but in addition to the pair of laterally positioned flanges, the fluid control adapter includes an adapter coupling feature in the form of magnets, which can magnetically couple the proximal adapter surface 232 with a distal body surface (not shown, but shown in FIGS. 1 and 6 at 224).

With respect to coupling the adapter coupling features that can be implemented for coupling the fluid control adapter along its proximal adapter surface to a distal body surface of a fluid directing body, there are many additional options that can be used. These adapter coupling features can likewise be used at the distal adapter surface (not shown, but shown in FIGS. 1, 3, 4, and 7) to couple the fluid control adapter to a second fluid control adapter or to an applicator tip. It is also noted that the fluid directing body and the applicator tip can also include coupling features that correspond with connection the adapter coupling feature of the fluid control adapter(s), but are referred to as “body coupling features” and “tip coupling features,” respectively. Discussion of the adapter coupling features is relevant to the types of coupling features that can be used on the fluid directing body and/or the applicator tip in connecting these with intervening fluid control adapters.

Example adapter coupling features that are connectable to body coupling features and/or tip coupling features include tongue or groove connectors, snaps, screws, clamps, pins, loop or hook fasteners, click fasteners, compression fittings, permanent magnets, electromagnets, temporary adhesives, temporary bonding compounds, electrostatic elements, spring locks, slide fasteners, or a combination thereof. This adapter coupling feature can be configured to connect with a corresponding body coupling feature or tip coupling feature. Typically, there is an adapter coupling feature at both its proximal adapter surface (to releasably or removably connect to the distal body surface of a fluid directing body) and its distal adapter surface (to releasably or removably connect to the proximal tip surface of an applicator tip).

In some examples, in addition to the various coupling features found on the fluid directing body, the fluid control adapter(s), and/or the applicator tip, there may also be electrical, optical, thermal, or mechanical connection features to provide functional interaction with one another (beyond simple fluidic connection). These connections can be integrated with the various coupling feature(s) or can be distinct structures relative to the coupling features. For example, there may be electrical contact pads on the various surfaces to make an electrical connection when the structures are fluidically coupled together. Alternatively, there may be fiber optics that pass through the various structures to allow for the passage of light from the fluid directing body all the way to the application surface when the various components are properly seated together.

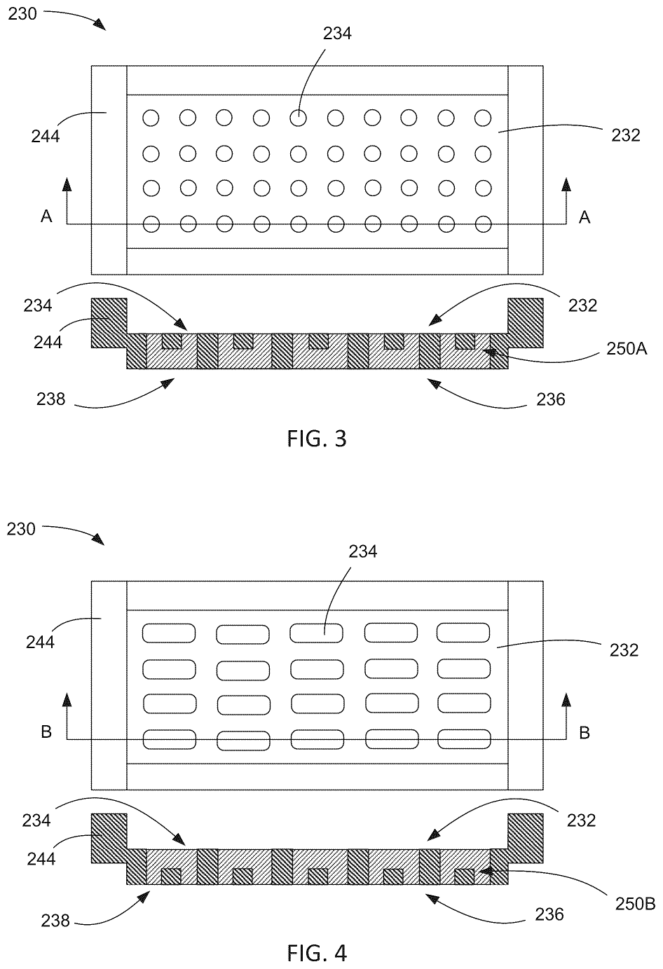

Referring now to FIG. 3 and FIG. 4, two example fluid control adapters 230 are shown. A top plan view is shown along with a cross-sectional view along section A-A in FIG. 3 and a top plan view is shown along with a cross-sectional view along section B-B in FIG. 4. Each of these two fluid control adapters include different fluid modification architectures or components. FIG. 3 illustrates an adapter in the form of a fluid combiner 250A and FIG. 4 illustrates an adapter in the form of a fluid divider 250B. The fluid control adapters shown include a proximal adapter surface 232 defining proximal adapter openings 234, as well as a distal adapter surface 236 defining distal adapter openings 238. In some examples, the arrangement shown in FIG. 4 could provide proximal adapter openings large enough for multiple distal body openings of a fluid directing body (not shown). Other arrangements for combining and/or dividing can likewise be arranged, e.g. from 4 distal body openings to 1 proximal adapter opening, from 8 distal body openings to 1 proximal adapter opening, from all distal body openings to 1 proximal adapter opening, from 1 distal body opening to 4 proximal adapter openings, from 1 distal body opening to 8 proximal adapter openings, from 1 distal body opening to all proximal adapter openings, and so forth. The same could be the case when connecting a first fluid control adapter to a second fluid control adapter, or a fluid control adapter to an applicator tip. Various combiner and/or divider fluid modification architectures could be implemented between any two fluidically connected surfaces.

Turning now to FIG. 5, an example modular fluid control adapter system 300 is shown that includes multiple stackable fluid control adapters 230 attached which are releasably attachable together to form a portion of a flow cell when combined with a fluid directing body (not shown) and an applicator tip 280. In this example, the multiple fluid control adapters each include a proximal adapter surface 232 that defines a plurality of proximal adapter openings 234. The multiple fluid control adapters also each include a distal adapter surface 236 that defines a plurality of distal adapter openings. Between one or more proximal adapter opening(s) and one or more distal adapter opening(s) are adapter microfluidic channels 240. Thus, fluid can be circulated from a fluid directing body (not shown) through a cascade of connected fluid control adapters and an application tip to deposit a substance on an application surface (not shown). The term “circulate” is used in this example as fluid can also be configured to return from the application surface through the applicator tip, through the cascade of fluid control adapters (in the reverse order) and back into or through the fluid directing body. Fluid may likewise be passed in a reverse flow path in instances where bi-directional flow may be useful or called for in a given application.

In additional detail, the modular fluid control adapter system 300 can include a first fluid control adapter (which can be any of the fluid control adapters shown in FIG. 5) with an adapter microfluidic channel fluidically connecting one or more proximal adapter openings defined by a first proximal adapter surface with one or more distal adapter openings defined by a first distal adapter surface. The modular fluid control adapter system can also include a second fluid control adapter (which can be any of the fluid control adapters positioned beneath the first fluid control adapter or can be the applicator tip 280) that includes a second adapter microfluidic channel fluidically connecting one or more proximal adapter openings defined by a second proximal adapter surface with one or more distal adapter openings defined by a second distal adapter surface. The first distal adapter surface is releasably sealable against the second proximal adapter surface to allow the flow of fluid from within the first adapter microfluidic channel to within the second adapter microfluidic channel. In additional detail, the first adapter microfluidic channel can interact mechanically, optically, chemically, biologically, or thermally with fluid when introduced therein in a manner that is different than how the second adapter interacts with the fluid.

While the fluid is being contained within or passing through the various fluid control adapters 230, the fluid may be acted upon by any of a number of fluid modification architectures or components, shown generally in FIG. 1 at 250. More specific fluid modification architectures or components are shown by way of example in the various fluid control adapters shown in FIG. 5. For example, the fluid modification architecture or component may be in the form of a fluid combiner 250A, a fluid divider 250B, a fluid redirector 250C, an electrical element(s) 250D, e.g., capacitors, resistors, inductors, piezoelectric components, antennas, etc., a thermocontroller 250E, e.g., heater, cooler, heat sink, etc., a valve 250F, e.g. a mechanical, a MEMS, a bubble, etc., a fluid sensor 250G, e.g., optical, chemical, MEMS, electrical, etc., a reagent 250H, a fluid mixer 250J or agitator, a fluid storage chamber 250K, a fluid blocker 250L, and/or the like. With respect to the fluid blocker, in some examples, a fluid control adapter may include one or more proximal adapter openings associated with unconnected channels that do not permit the fluid to pass into the applicator tip. Here, the fluid blocker is shown at a proximal adapter surface, but it is also noted that the fluid storage chamber shown at 250K and the fluid redirector shown at 250C also may provide fluid blocking, as the fluid is not permitted to pass therethrough when storing and/or redirecting fluid back toward the fluid directing body at this location.

As can be seen from this arrangement, various fluid flow paths can be assembled by a system user by simply selecting appropriate fluid control adapters and then stacking them appropriately to generate the function that the user intends. This “multi-layering” approach can be particularly useful with biosensors and other microanalysis platforms where a substance is applied to an application surface for chemical and/or biological analysis. To illustrate, an application surface is often prepared in advance for detection of a substance. Thus, in some examples, one or more fluid control adapters can be selected for use in the fluidic preparation of an application surface so that the assay can be effectively carried out, and then one or more fluid control adapters can be selected for applying a substance to the application surface that was previously prepared. As a more specific example, an application surface, such as a sensor array, e.g. surface plasmon resonance substrate, can be interrogated with a fluid carrying a capture substance that is known to capture a specific analyte of interest. The capture substance can be applied using one of the fluid control adapters or multiple fluid control adapters as described herein. Then, a second fluid that may be carrying the analyte of interest can be applied to the spot where the capture substance has been applied using a different fluid control adapter to process the fluid prior to application, or to carry out some other function. For example, the fluid control adapter may be in the form of an applicator tip with a sensor positioned at the tip to sense whether the analyte was present based on whether it was chelated or complexed with the capture substance. Alternatively, the capture substance may be applied as very large spots using a fluid control adapter(s) with one or more fluid combiners, e.g., combining fluid from many microchannels into a single large flow chamber of an applicator tip, and then the fluid containing the potential analyte (or multiple fluids that may contain the analyte) can use a different single or group of fluid control adapters for application to the large spot of the capture substance, or vice versa. In further detail, the versatility of the modular microfluidic flow cell arrays of the present disclosure makes it possible to integrate this system with a variety of analysis platforms, and can be particularly useful for applications that utilize internal referencing. For example, sensors can be used to detect substance properties and/or interactions with a reference region compared to other regions of interest after applying a substance(s) on the application surfaces. Furthermore, the substance spotter systems of the present disclosure can include computers, networks, and/or other processing device for analyzing signals from various sensor and/or control fluid modification architecture or components.

Referring now to FIG. 6 and FIG. 7, an example modular microfluidic flow cell array is shown, with two views of a fluid directing body 210 shown in FIG. 6 (front plan view and distal end view, respectively) and multiple fluid control adapters 230 shown in FIG. 7. The modular microfluidic flow cell array as shown in this example (and which may be relevant to other examples) can include the fluid directing body defining a plurality of body microfluidic channels fluidically connecting source fluid openings with distal body openings at a distal body surface. Referring more specifically to FIG. 6, the fluid directing body of this example is shown as including many source fluid openings, with a specific pair of source fluid openings 211 or fluid ports identified as a first source fluid opening 212 and a second source fluid opening 214. Also shown are many body microfluidic channels, including a pair of body microfluidic channels 215 identified more specifically as including a first body microfluidic channel 216 and a second body microfluidic channel 218. The various body microfluidic channels in this example can thus be accessed for introduction or retrieval of fluid from the source fluid openings. The body microfluidic channels also provide fluid channeling through the fluid directing body to respective distal body openings 226 defined by a distal body surface 224. In this particular example, a body coupling feature 228 is included in the form of four magnets which are oriented with their south pole along the distal body surface and are also shown to provide a body coupling feature 228 for attachment to a fluid control adapter (not shown in FIG. 6, but shown in FIG. 7).

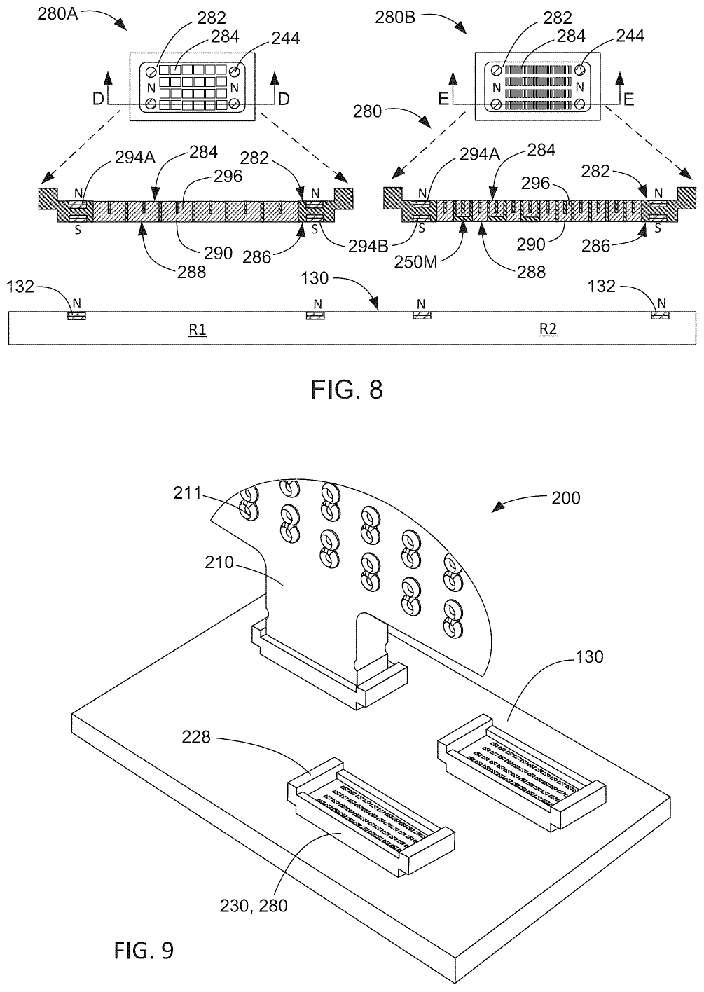

Referring now more specifically to FIG. 7, two fluid control adapters 230 are shown. However, it is noted that one of the fluid control adapters may alternatively also be in the form of an applicator tip 280, which includes multiple flow chambers 290. If this fluid control adapter is in the form of an applicator tip, then inflow (or outflow) architecture defining several flow chambers is shown at the lower fluid control adapter, and outflow (or inflow), respectively may occur through one or more of its adjacent proximal adapter openings along the row of proximal adapter openings in alignment with Row 298. In other words, as applicator tips typically include an inflow and an outflow channel (or vice versa in instances of bi-directional flow), rather than inflow and outflow being present in a right to left or left to right arrangement (as shown in FIG. 8 by way of example), it is understood that inflow and outflow would occur in this design in a front to back arrangement, with its corresponding inflow or outflow channel being obscured due to the right to left cross-sectional view as shown in FIG. 7. On the other hand, if the lower fluid control adapter was not an applicator tip, but rather an intermediate fluid control adapter with a separate applicator tip (not shown), then this particular arrangement would not include a corresponding inflow or outflow channel, similar to that shown and described at 230 in FIGS. 1-5.

As shown in this example, a first fluid control adapter is shown along its proximal adapter surface 232 which defines a plurality of proximal adapter openings 234. The first fluid control adapter also includes an adapter coupling feature 244 in the form of four magnets which are oriented with their north pole along the proximal adapter surface for coupling with the four south pole facing magnets of the distal body surface of the fluid directing body (FIG. 6). Notably, the proximal adapter openings of FIG. 7 are of various sizes, and all of them are larger than the size of the distal body openings shown at 226 of FIG. 6. Thus, when the fluid directing body and the first fluid control adapter are connected together magnetically, in some instances, four distal body openings will be fluidically connected with one proximal adapter opening, and in other instances, eight distal body openings will be fluidically connected with one proximal adapter opening. When the distal body surface of the fluid directing body shown in FIG. 6 becomes connected with the proximal adapter surface of the fluid control adapter of FIG. 7, the two respective surfaces will seal the body microfluidic channels with the adapter microfluidic channels to allow for fluid flow without substantial crosstalk.

The first fluid control adapter 230 of FIG. 7 in this example includes three different types of fluid modification architectures or components. For example, two fluid control adapters 230 (or one fluid control adapter 230 and one applicator tip 280) are shown in cross-section along cross-section C-C, illustrating various types of adapter microfluidic channels 240 or in the case of an applicator tip, various flow chambers 290. The upper fluid control adapter can include a one channel to four channel fluid divider 250B, two of the adapter microfluidic channels shown include a one channel to two channel fluid divider, and one of the adapter microfluidic channels shown includes a fluid mixer 250J in the form of a baffle. The first fluid control adapter also includes adapter coupling features 244 with four south pole facing magnets (two shown in cross-section) for attaching to a second fluid control adapter 230, which in this instance is also an applicator tip 280.

The second fluid applicator 230, or applicator tip 280, in this example, includes tip microfluidic channels 296 that are aligned with the adapter microfluidic channels 240 at its distal adapter openings 238, which is a typical way that an applicator tip would be used to channel fluids to an application surface 130. However, in this particular applicator tip, a fluid modification architecture or component is shown in cross-section, which is also a fluid mixer 250J. Thus, the fluid mixer of the first fluid control adapter and the second fluid mixer of the applicator tip can work together in series and provide additional mixing properties due to the narrowing and expansion of the fluid flow, together creating a more tortious pathway prior to application of a substance spot onto the application surface.

In the example shown, the application surface 130 may be equipped with an application surface coupling feature 132, which in this example is in the form of four electromagnets (two shown in cross-section), which can be turned on and off for fluidly connecting the applicator tip 280 with the application surface when electrical current is flowing, and then by turning off the electrical connection to release the applicator tip when the applied substance spot, e.g., printed substance spot, or other fluid flow contacting the application surface is complete. This coupling feature may be magnetically coupled to a tip coupling feature 294 of the distal tip surface 286 of the applicator tip. Notably, in this example, there is also a tip coupling feature shown at the proximal tip surface for connection with the adapter coupling feature of the distal adapter surface 236.

In further detail, the first fluid control adapter 230 can include an adapter microfluidic channel 240 fluidically connecting one or more proximal adapter openings 234 defined by a first proximal adapter surface 232 with one or more distal adapter openings 238 defined by a first distal adapter surface 236. The first proximal adapter surface can be removably connectable with the distal body surface of the fluid directing body (shown in FIG. 6 at 210). The applicator tip 280 can likewise be removably connectable with the first distal adapter surface of the first fluid control adapter. The fluid directing body, the first fluid control adapter, and the applicator tip are fluidically connected and substance applied to an application surface occurs as fluid passes from the fluid directing body, through the first fluid control adapter, and through the applicator tip depositing the substance to the application surface. In some examples, the first adapter microfluidic channel can interact mechanically, optically, chemically, biologically, or thermally with fluid during at least a portion of the time that fluid is contained therein or passing through the first fluid control adapter. For example, the first adapter microfluidic channel may include mechanical architecture to introduce fluid contained within or passing through the first fluid control adapter to fluid dividing, fluid combining, fluid mixing, fluid sampling, fluid trapping, air bubble separation, or a combination thereof; an electrical component to introduce fluid contained within or passing through the first fluid control adapter to electrical current, capacitance, electrophoresis, dielectrophoresis, electroshock, electrospray, electromagnetic frequency, electromagnetic field, resistance, induction, piezoelectric interaction, or a combination thereof; a valve; a sensor to sense a property of the fluid, wherein the sensor is an optical sensor, a chemical sensor, a microelectromechanical system (MEMS) sensor, an electrical sensor, a biological sensor, an NMR sensor, a dielectrophoretic sensor, or a combination thereof; a dry reagent or a fluid reagent to interact with fluid contained within or passing through the first fluid control adapter; a thermocontroller to heat, cool, or control the temperature of the fluid contained within or passing through the first fluid control adapter; a particle or cellular capture or sorting apparatus; or the like.

In other examples, in addition to the first fluid control adapter 230 and the applicator tip 280, there may be an alternative “second” fluid control adapter, such as shown in FIG. 5 where there are first, second, third, and fourth fluid control adapters shown. In such examples, the second fluid control adapter can have a second adapter microfluidic channel fluidically connecting one or more proximal adapter openings defined by a second proximal adapter surface with one or more distal adapter openings defined by a second distal adapter surface. The second fluid control adapter can be positioned or positionable between the first fluid control adapter and the applicator tip and/or can be interchangeable with the first fluid control adapter. For example, the second fluid control adapter can be positioned between the first fluid control adapter and the applicator tip such that the second adapter microfluidic channel fluidically connects the first adapter microfluidic channel with an applicator flow channel of the applicator tip. In other examples, the first adapter microfluidic channel can interact mechanically, optically, chemically, biologically, or thermally with fluid during at least a portion of the time that fluid is contained within or passing through the fluid control adapter. Examples of this interaction can occur by one or more of the adapter coupling features previously described. The second adapter microfluidic channel can also interact mechanically, optically, chemically, biologically, or thermally with fluid during at least a portion of the time that fluid is contained within or passing through the fluid control adapter. In this example, the first adapter microfluidic channel and the second adapter microfluidic channel may interact differently with the fluid relative to one another.

In additional examples related to that shown in FIGS. 1-2D, and FIGS. 6 and 7, connection and disconnection of the fluid directing body, the first fluid control adapter, and the applicator tip can occur by automation. For example, a second fluid control adapter may be exchanged with the first fluid control adapter by automation and/or the second fluid control adapter can be connected between the first fluid control adapter and the applicator tip by automation. Automation can be carried out using the substance spotter system 100 shown in FIG. 1, or by any other automation equipment.

In further detail, connection between the distal body surface of the fluid directing body and the proximal adapter surface of the first fluid control adapter can occur in a manner that makes an electrical, optical, thermal, or mechanical connection that can be translated from one structure to the next. Likewise, an electrical, optical, thermal, or mechanical connection can be made between the first distal adapter surface of the first fluid control adapter with the applicator tip or a second fluid control adapter positioned between the first fluid control adapter and the applicator tip.

Referring now to FIG. 8, multiple applicator tips 280A and 280B are shown which are suitable for being fluidically connected to the fluid directing body shown and described by way of example in connection with FIG. 6 at 210. The multiple applicator tips of FIG. 8 in combination with the fluid directing body of FIG. 6 provide an example of a modular microfluidic flow cell array. In some examples, one or more fluid control adapters that are not also applicator tips can be included in the modular microfluidic flow cell array or system. In this example, an application surface 130 is shown with two regions, namely region R1 and region R2. Either or both of these applicator tips can be configured to apply substance spots or otherwise treat the application surface at either or both of these locations, for example.

In this example as shown in FIG. 8, there are multiple applicator tips 280A and 280B shown. The applicator tips may be configured to be removably coupled directly to the distal body surface (not shown, but shown by way of example in FIG. 6 at 224) of the fluid directing body. Whether or not there are intervening fluid control adapters, in this example, there may be multiple applicator tips that are exchangeable with one another. In some examples, the exchangeable applicator tips may be the same, e.g., replacing applicator tip 280A with a replacement applicator tip 280A or replacing applicator tip 280B with a replacement applicator tip 280B. In other examples, the exchangeable applicator tip may be a second applicator tip that may include a different material or have a different configuration, e.g. replacing applicator tip 280A for applicator tip 280B (or vice versa).

In further detail, the applicator tip 280A or 280B may include a proximal tip surface 282 defining proximal tip openings 284 and a distal tip surface 286 defining distal tip openings 288 fluidically connected to the proximal tip openings via tip microfluidic channels 297. The distal tip openings can be arranged to provide fluid communication between the applicator tip and an application surface 130 when the distal tip surface is contacted therewith. The applicator tip may also include a distal tip coupling feature 294A at or adjacent to the proximal tip surface. Thus, the distal tip coupling feature can be removably connectable to the fluid directing body (not shown, but shown at 210 in FIG. 6) to provide fluidic communication between the distal body openings (shown at 226 in FIG. 6) with the proximal tip openings. In some examples, the applicator tip may also include a distal tip coupling feature 294B at or adjacent to the distal tip surface that can be removably connectable to application surface 130, such as at an application surface coupling feature 132.

In further detail, as mentioned, the modular microfluidic flow cell array 200 of this example can include multiple applicator tips 280A or 280B of the same type, or of different types 280A and 280B, as shown. Thus, in some examples, in addition to the (first) applicator tip 280A, a second applicator tip 280B can be exchangeable with the applicator tip, and can include a second proximal tip surface 282 defining second proximal tip openings 284. The second applicator tip (shown at 280B) can also include a second distal tip surface 286 defining second distal tip openings 288 fluidically connected to the second proximal tip openings via tip microfluidic channels 296. The second distal tip openings can be arranged to provide fluid communication between the second applicator tip and the application surface 130 when the second distal tip surface is contacted therewith. As with the (first) applicator tip shown at 280A, the second applicator tip 280B can also include a second adapter coupling feature at or adjacent to the second proximal tip surface, wherein the second distal tip coupling feature 294A may be removably connectable to the fluid directing body (shown at 210 in FIG. 6) to provide fluidic communication between the distal body openings (shown at 226 in FIG. 6) with the second proximal tip openings. In some examples, the second applicator tip 280B may also include a distal tip coupling feature 294B at or adjacent to the distal tip surface that can be removably connectable to application surface 130, such as at an application surface coupling feature 132.

In additional detail, such as when the first applicator tip 280A is different than the second applicator tip 280B, the differences can be based on a variety of modifications, including those not shown in FIG. 8. For example, the second applicator tip can be adapted to generate a different spotted pattern compared to the (first) applicator tip. In other examples, the second applicator tip can be adapted to generate a different number of spots compared to the applicator tip. The second applicator tip can be adapted to generate different spot shapes, different spot sizes, or different spot orientations compared to the applicator tip, e.g., elongated spots or lines of orthogonal orientations relative to one another, square vs rectangle shapes, triangle vs. round shapes, etc., and small vs. larger spot footprints, etc. In other examples, the second applicator tip can include a different material that is harder or softer compared to the applicator tip. The harder or softer material may be throughout the applicator tips(s) or may be concentrated at either the distal tip surface and/or proximal tip surface. In some instances, a softer material may provide a better seal when joining with other structures under certain circumstances than a harder material, and vice versa. Furthermore, the second applicator tip can be manufactured to have a different thickness compared to the applicator tip. A thicker second applicator tip may be more suitable for reaching recessed application surfaces in some examples, or alternatively, a thicker applicator tip can be sized to have the same thickness as the (first) applicator tip attached to the fluid directing body (not shown in FIG. 8, but shown at least in FIGS. 1-2D and 6) with an intervening fluid control adapter (not shown in FIG. 8, but shown at least in FIGS. 1-7).

In further detail, the applicator tip(s) 280A or 280B may include a fluid modification architecture or component that interacts mechanically, optically, chemically, biologically, or thermally with fluid during at least a portion of the time that fluid is contained within or passing through the applicator tip or the second applicator tip, depending on which one is fluidically connected to the fluid directing body (not shown). A “fluid modification architecture or component” is defined similarly as when present in a fluid control adapter, but in this context, it is present in one or more of the applicator tips. For example, the fluid modification architecture or component may be defined as something other than exterior walls that laterally circumscribe the general shape of the tip microfluidic channels 296 and/or the flow chamber 290.

In accordance with more specific examples, the fluid modification architecture or component may include a mechanical architecture to introduce fluid contained within or passing through the applicator tip or the second applicator tip to fluid dividing, fluid combining, fluid mixing, fluid sampling, fluid trapping, air bubble separation, or a combination thereof. In other examples, the fluid modification architecture or component may include an electrical component to introduce fluid contained within or passing through the applicator tip or the second applicator tip to electrical current, capacitance, electrophoresis, dielectrophoresis, electroshock, electrospray, electromagnetic frequency, electromagnetic field, resistance, induction, piezoelectric interaction, or a combination thereof. In other examples, the fluid modification architecture or component may include a valve. In still other examples, the fluid modification architecture or component may include a sensor to sense a property of the fluid contained within or passing through the applicator tip or the second applicator tip, wherein the sensor is an optical sensor, a chemical sensor, a microelectromechanical system sensor, an electrical sensor, a biological sensor, an NMR sensor, a dielectrophoretic sensor, a capacitance sensor, or a combination thereof. In still other examples, the fluid modification architecture or component can include a dry reagent or a fluid reagent to interact with fluid contained within or passing through the applicator tip or the second applicator tip. In other examples, the fluid modification architecture or component can include a thermocontroller, e.g., cooler, heater, temperature stabilizer, heat sink, etc.

In accordance with other examples of the present disclosure, even though FIG. 8 includes example applicator tips 280A and 280B as well as an example applicator surface 130, it is understood that either or both of these example applicator tips, or any others described herein (including those with fluid modification architectures or components described in the context of the fluid control adapters), can be joined with any of the other structures described herein as part of a modular microfluidic flow cell array or related system. For example, the applicator tips can be directly fluidically coupled to a fluid directing body, such as that shown by way of example in FIG. 6 at 210, or the applicator tips can be coupled indirectly to a fluid directing body through one or more fluid control adapters, as shown by way of example at least in FIGS. 1-7 at 230.

Referring now to FIG. 9, a portion of a fluid directing body 210 is shown that again includes source fluid openings 211 and a body coupling feature 228 (which is a compression coupler in this example). However, in this example, the fluid control adapter 230, which is also in the form of an applicator tip 280, is shown as being integrated with or attached to an application surface 130. Notably, there are multiple applicator tips integrated with or attached to the application surface, enabling the fluid directing body (and any other intervening fluid control adapters) to be removably connected with the applicator tip at different locations by movement of the fluid directing body. For example, a substance spotter system can include a positioning sensor 154 and a force sensor 156 with a z-axis positioner 150 and an xy-axis positioner 152, as shown in FIG. 1, can be used to align, engage with (insert), and disengage from (withdraw) the applicator tip that is integrated with or attached to the application surface. The applicator tips integrated with or attached to the application surface can be the same in configuration or different in configuration. In some examples, the applicator tip(s) may be attached to the application surface prior to use, and then may remain on the applicator surface after use in applying substance spots or interrogation. In other examples, after applying substance spots or interrogation, the fluid directing body (or any intervening fluid control adapter) can provide a strong enough connection with the applicator tip to detach the applicator tip from the applicator surface. An example of this may include the use of a stronger magnetic attraction or a stronger friction fit/compression coupling than the strength of attachment between the applicator tip and the application surface, the use of electromagnets at the application surface that may be turned off so that the application tip can be removed, or the like.

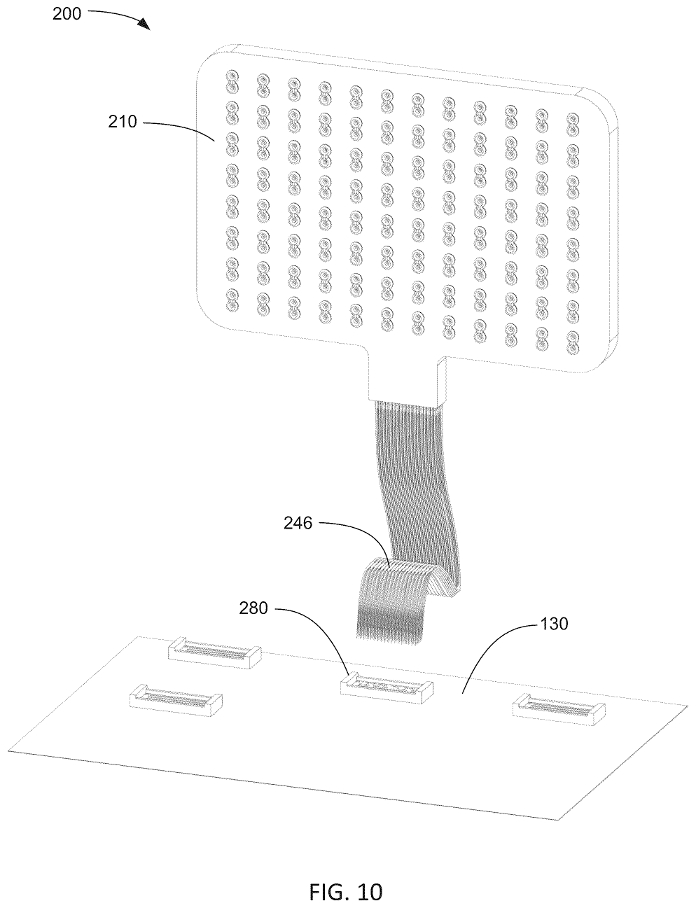

Referring now to FIG. 10, a similar arrangement of a fluid directing body 210, an application surface 130, and a plurality of integrated or attached applicator tips 280 are shown. However, in this example, an array of elongated hollow conduit 246 is used to bridge the fluid directing body and the applicator tip. In these examples, the term “elongated” refers to flow channels that are longer in length (in the direction of flow path) than the length of the tip microfluidic channels and any adapter microfluidic channels of the modular microfluidic flow cell array. The array of elongated hollow conduit can be in the form of a ribbon of flexible hollow conduit, e.g., flexible polymeric microchannels, or the array of elongated hollow conduit may be rigid, e.g., array of microneedles. The array of elongated hollow conduit may be integrated with the fluid directing body, or alternatively, it may be detachable from the fluid directing body and act as a fluid control adapter. A function provided by the elongated array of hollow conduit may be to allow for flexibility of attachment, longer flow paths to carry out cooling or heating, flow paths for sensing a fluid property, etc. Regarding flexibility of attachment, the array of elongated hollow conduit can be configured to preserve the microfluidic connection to the applicator tip attached to or integrated with the application surface even when the fluid directing body is moved in an x-direction, a y-direction, a z-direction, or a combination of directions thereof. One advantage of having the applicator tip connected directly to the application surface is that it can remove the possibility of contamination, dust particles, etc., occurring between the applicator tip and the application surface.

Turning now to various methods of the present disclosure, methods of treating an application surface for analysis of a substance can include docking a first modular microfluidic flow cell array on an application surface and flowing a first fluid through the first modular microfluidic flow cell array including through the first fluid control adapter. In this example, the first modular microfluidic flow cell array can include a fluid directing body fluidly connected to a first fluid control adapter. The method can further include undocking the first modular microfluidic flow cell array from the application surface, and removing the first fluid control adapter from the fluid directing body. In further detail, the method can include fluidly connecting a second fluid control adapter to the fluid directing body to form a second modular microfluidic flow cell array, docking the second modular microfluidic flow cell array on the application surface, and flowing a second fluid through the second modular microfluidic flow cell array including through the second fluid control adapter. Structures suitable for carrying out this method are shown by way of example at least in FIGS. 1-7.

In another example, methods of applying substances for analysis to an application surface can include fluidly connecting a fluid directing body with a first applicator tip to form a first microfluidic flow cell array, docking the applicator tip of the first modular microfluidic flow cell array on an application surface, flowing a first fluid through the fluid directing body and the first applicator tip, and disconnecting the fluid directing body from the first applicator tip. In further detail, the method can include fluidly connecting the fluid directing body with a second applicator tip to form a second microfluidic flow cell array, docking the second applicator tip of the second modular microfluidic flow cell array on the application surface, and flowing a second fluid through the fluid directing body and the second applicator tip. In this example, flowing the first fluid and flowing the second fluid results in application of at least one set of substance spots on the application surface. Structures suitable for carrying out this method are shown by way of example at least in FIGS. 9-10. In this particular example, the first applicator tip, the second applicator tip, or both may be permanently joined with the application surface, or they may be impermanently joined with the application surface, but are joined sufficiently to withstand pulling pressure applied when disconnecting from the fluid directing body. In some examples, the first applicator tip, the second applicator tip, or both, and the application surface may be in the form of a consumable. The first applicator tip, the second applicator tip, or both may be joinable with an array of hollow conduits, for example, such as a rigid array of microneedles or a ribbon of flexible hollow conduit. With flexible hollow conduit, the first applicator tip, the second applicator tip, or both, can be configured to preserve the microfluidic connection to the fluid directing body even when the fluid directing body or the application surface is moved in an x-direction, a y-direction, a z-direction, or a combination of directions thereof. In some examples, flowing the first fluid or flowing the second fluid may not be application of substance spots, but rather is for application surface preparation or substance spot treatment. Examples include deposition spot washing, microfluidic channel priming, generating a reaction, removing materials, swapping materials, manipulating cells, e.g., delivery, feeding, exposure to flow conditions, etc., or a combination thereof.

With respect to each of these methods, in some examples, a first fluid and a second fluid may be used and may be the same fluid, and in other examples, the first fluid and the second fluid may be different. In other examples, there may be two fluid control adapters and the first fluid control adapter may interact differently with the fluid(s) compared to the second fluid control adapter. In other examples, the first fluid control adapter, the second fluid control adapter, or both, are in the form of a first applicator tip, a second applicator tip, or both, respectively.

Methods of the present disclosure that utilize multiple fluid control adapters that are separate and distinct from one or more applicator tips can further include fluidly connecting a first applicator tip to the first fluid control adapter to deposit the first group of substance spots on the application surface via the first applicator tip, fluidly connecting a second applicator tip to the second fluid control adapter to deposit the second group of substance spots on the application surface via the second applicator tip, or both.

Deposition of substance spots to an application surface can include depositing a first group of substance spots and a second group of substance spots at overlapping interstitial locations on the application surface. For example, the overlapping interstitial locations may be partially overlapping with one or both of the first group of substance spots or the second group of substance spots being applied to unspotted space on the application surface, or they may be fully overlapping. In another example, the method can include depositing a first group of substance spots and a second group of substances spots at non-overlapping interstitial locations on the application surface.

In further detail, the modular microfluidic flow cell arrays of the present disclosure provide for the ability to apply substance spots with individually addressed flow chambers, and furthermore, a large number of spot arrays can be addressed in parallel. Constant fluid flow to apply or interrogate the application surface with a substance can be maintained for an extended period of time to allow spotted areas to build a high-density spot. This technique allows for much higher signals to be generated than when standard concentrations are used with traditional spotters. These higher signals can increase the signal-to noise ratio of the applied substance compared to background or other signals, thereby allowing for improved data collection. Thus, even relative low concentrations of substances in fluids may also be used with the spotter and still yield probative results, making the assaying of fluids with scant material possible with more reliable results. Some examples of assays that may be conducted using the modular microfluidic flow cell arrays of the present disclosure include fluorescence spectroscopy, chemiluminescence detection, color-staining, other optically-based microarray sensing technologies, radiometrics, etc. In other examples, probes and/or target compounds can be applied to a previously deposited spot or area of spots until a high-density spot has been created. Examples of probes that may be flowed over a surface include: proteins; nucleic acids, including deoxyribonucleic acids (DNA) and ribonucleic acids (RNA); cells; peptides; lectins; modified polysaccharides; synthetic composite macromolecules; functionalized nanostructures; synthetic polymers; modified/blocked nucleotides/nucleosides; synthetic oligonucleotides; modified/blocked amino acids; fluorophores; chromophores; ligands; chelates; haptens; drug compounds; antibodies; sugars; lipids; liposomes; tissue; viruses; any other nanoscale or microscale objects; and any combinations thereof.

The flow of fluid substance or fluids containing substances over an application surface 130 can provide an opportunity for a substance to bind to, adhere with, adsorb on, etc., to the application surface, depending on the chemistry involved in the system, e.g., the fluid, substance, application surface, etc. Binding, adhering, adsorbing, etc., can occur between the substance and the application surface as designed and/or in a non-specific manner in some instances. By way of example, non-specific binding can describe binding of a molecule of interest to a surface not specifically activated for such binding. A more typical example of non-specific binding refers to binding by species of molecules beside the species of interest. These examples are mentioned by way of illustration, and are not intended to be limiting. In either case, nonspecific binding of a substance can produce a binding-related signal that may be detectable by an assay technique, but may or may not provide any useful information.

Use of modular microfluidic flow cell arrays of the present disclosure can be implemented in a way that promotes low cross-talk and low background noise, internal referencing, and/or subtraction of unwanted signals due to nonspecific binding and substrate surface. For example, an applicator tip 280 can be utilized to include discrete and well-defined spots corresponding to the positions of the flow chambers, as well as unspotted areas surrounding individual spots providing little to no contact adjacent to each spot. These unspotted areas (or areas “printed” with another substance) can provide a reference against which to evaluate any binding that occurs at the spot location(s). Accordingly, the present disclosure includes the possibility of carrying out microanalysis using unspotted areas as a reference. For example, an effective way of using these spaces as a reference can include interrogating a spot and an adjacent unspotted space with the same sample solution. In this instance, both locations can be analyzed in parallel. Any binding that occurs in the spot can be compared to any binding detected in the unspotted space. During interrogation, any binding that is not specific to the spotted capture material may occur in both the spot locations(s) and the unspotted space. Such non-specific binding can be accounted for by generating a signal that corresponds to binding density and then comparing the signal from the applied substance spot with that with the signal. In a more particular approach, the signal from the unspotted space is subtracted from the signal detected in the spotted portions. This approach can also be used to correct for any artefactual signal that is not associated with binding but is rather produced by the substrate surface itself. In further detail regarding internal referencing, correction(s) can be made more effective if an applied substance spot and an unspotted space can often be interrogated by the same bolus of sample fluid. This ensures that both surfaces are contacted by the same substance, reducing concern arising from possible variation among samples. Accordingly, the present disclosure provides methods and systems for microanalysis with correction using an internal reference. Furthermore, any binding at an applied substance spot can be corrected for non-specific binding.

The modular microfluidic flow cell arrays 200 of the present disclosure may be used to produce any of a number of two-dimensional arrays of substance spots, and in some instances, can be used to fabricate microarrays with any practical number of defined spots, where each spot is individually tailored to a specific deposition density. The modular microfluidic flow cell arrays of the present disclosure may also be used to sequentially chemically process individual spots, provide for sequential deposition and testing, perform layer-by-layer self-assembly (LBL) to build up spot concentration, e.g., multiple layering and washings on the spotted area may be performed simply by changing the fluid/substance that is flowed over a spot, etc. The flow cells within the modular microfluidic flow cell array can likewise be modified by flowing the appropriate material through the spotter that modifies the properties of the flow cells in any of the various assembled structures or prior to assembly of the modular microfluidic flow cell array, for example. Surface modification of the internal walls of any of the microfluidic channel(s) of a flow cell may be performed using any of a number of solutions, such as BSA (bovine serum albumin) to reduce binding of a substance. In some examples, the modular microfluidic flow cell array, or a portion thereof, may be disposable, which could be beneficial to avoid contamination issues.

In other examples, methods can include removing a first fluid control adapter from the fluid directing body and fluidly connecting a second fluid control adapter to the fluid directing body carried out by mechanical disengagement of the fluid directing body with the first fluid control adapter and mechanical engagement of the fluid directing body with the second fluid control adapter. In other examples, methods can include removing the first fluid control adapter from the fluid directing body and fluidly connecting a second fluid control adapter to the fluid directing body carried out by magnetic disengagement of the fluid directing body with the first fluid control adapter and magnetic engagement of the fluid directing body with the second fluid control adapter. In other examples, methods can include removing the first fluid control adapter from the fluid directing body and fluidly connecting a second fluid control adapter to the fluid directing body carried out by electrostatic force disengagement of the fluid directing body with the first fluid control adapter and electrostatic force engagement of the fluid directing body with the second fluid control adapter. For example, removing the first applicator tip from the first fluid control adapter and fluidly connecting the second applicator tip to the second fluid control adapter may be carried out by: mechanical disengagement of the first fluid control adapter with the first applicator tip and mechanical engagement of the second fluid control adapter with the second applicator tip; magnetic disengagement of the first fluid control adapter with the first applicator tip and magnetic engagement of the second fluid control adapter with the second applicator tip; electrostatic force disengagement of the first fluid control adapter with the first applicator tip and electrostatic force engagement of the second fluid control adapter with the second applicator tip; adhesive disengagement with a non-permanent adhesive that can be adhered, disconnected, and then reconnected adhesively again; or the like.

In some examples, a first modular microfluidic flow cell array and the second modular microfluidic flow cell array can be arranged to deposit different patterns of substances on an application surface. In other examples, interrogation fluid can be applied to a plurality of spots or all spots of a first group of substance spots, a second group of substance spots, or both. The interrogation fluid may also be applied on unspotted space on the application surface surrounding the plurality of spots or all of the spots of the first group of substance spots, the second group of substance spots, or both, which as mentioned may be useful for carrying out internal referencing. The interrogation fluid may be applied using an interrogation applicator tip that is fluidically connectable directly or indirectly to the fluid directing body in some examples, e.g., the interrogation applicator tip can be fluidically connectable and disconnectable by mechanical, magnetic, or electrostatic force engagement and disengagement.

In other examples, methods may include depositing a first group of substance spots, a second group of substances spots, or both to an application surface, and also associating location data with the first group of substance spots, the second group of substance spots, or both by engaging a location sensor identifier with the application surface, the first modular microfluidic flow cell array, the second modular microfluidic flow cell array, the first group of substance spots, the second group of substance spots, or a combination thereof. Thus, the first modular microfluidic flow cell array, the second modular microfluidic flow cell array, or both, may include data collection hardware or indicia to collect data related to the deposition of the first group of substance spots, the second group of substance spots, or both. The data collection hardware or indicia may include, for example, an RFID tag, a 2D barcode, an electrical contact, or optics, wherein the data collection hardware is associated with a processor or processors associated with the first modular microfluidic flow cell array, the second modular microfluidic flow cell array, the application surface, or a combination thereof. A processor or processors can be configured to convey adjustments to an operation(s) selected from changing fluid flow parameters, expanding or restricting ports, contact pressure with the application substrate, temperature adjustment, valve operation, activating sensors, electromechanical manipulations, acoustic manipulations, optical manipulations, or a combination thereof. Furthermore, the processor or processors are in the form of an internal chip or chips. An internal chip or chips may be located on the first modular microfluidic flow cell array, the second modular microfluidic flow cell array, or both at a location or locations that contact the application surface when the first group of substance spots, the second group of substance spots, or both are being applied thereto, and/or the internal chip or chips may include an interlocking feature creating a fluid-tight seal with the application surface or withstanding pulling forces associated with release from the application surface.

In some examples, the fluid directing body, the first fluid control adapter, the second fluid control adaptor (inclusive of any type of applicator tip), or a combination thereof may include chamfers or guidance indicators to enhance fluidically coupling when engaging the fluid directing body with the first fluid control adapter, the second fluid control adaptor, or both. In other examples, whether there are chamfers or other guidance indicators or not, the connection and disconnection of the fluid control adapters to one another, to the fluid directing body, and/or the applicator tip may be carried out by automation.

With respect to fluid interaction with fluid modification architecture or components that may be included with the fluid control adapters and/or application tips, example functionalities may include thermally interacting with fluid contained within or passing through the first fluid control adapter, the second fluid control adapter, or both; mechanically interacting with fluid contained within or passing through the first fluid control adapter, the second fluid control adapter, or both, by fluid dividing, fluid combining, fluid mixing, fluid sampling, fluid trapping, air bubble separation, or a combination thereof; electrically interacting with fluid contained within or passing through the first fluid control adapter, the second fluid control adapter, or both, using electrical current, capacitance, electrophoresis, dielectrophoresis, electroshock, electrospray, electromagnetic frequency, resistance, induction, piezoelectric interaction, acoustics, or a combination thereof; sensing a fluid or substance property contained within or passing through the first fluid control adapter, the second fluid control adapter, or both, using an optical sensor, a chemical sensor, a microelectromechanical system (MEMS) sensor, an electrical sensor, a biological sensor, an acoustic sensor, a pressure sensor, or a combination thereof; chemically interacting with fluid contained within or passing through the first fluid control adapter, the second fluid control adapter, or both, using a first adapter microfluidic channel by contacting the fluid with a dry reagent or a fluid reagent contained therein.

### Embodiments

In accordance with the disclosure herein, the following examples are illustrative of several embodiments of the present technology.

1. A fluid control adapter, comprising:
a proximal adapter surface including an array of proximal adapter openings to receive fluid from a fluid directing body when fluidly connected therewith;
a distal adapter surface including an array of distal adapter openings to pass fluid to an applicator tip when fluidly connected therewith;
an adapter volume defining a plurality of adapter microfluidic channels individually fluidically connecting one or more proximal adapter openings with one or more distal adapter openings; and
a fluid modification architecture or component within the adapter volume to interact with fluid contained within or passing through the one or more of the adapter microfluidic channels.

2. The fluid control adapter of example 1, further comprising a first adapter coupling feature at the proximal adapter surface that is removably connectable to the fluid directing body.

3. The fluid control adapter of example 2, wherein the first adapter coupling feature is also removably connectable to a second fluid control adapter positionable between the fluid directing body and the proximal adapter surface of the fluid control adapter.

4. The fluid control adapter of example 2, wherein the first adapter coupling feature includes a feature selected from a tongue or groove connector, a snap, a screw, a clamp, a pin, a loop or hook fastener, a compression fitting, a permanent magnet, an electromagnet, a temporary adhesive, a temporary bond, an electrostatic element, a click fastener, a spring fastener, or a slide fastener, and wherein the first adapter coupling feature is adapted to connect with a body coupling feature.

5. The fluid control adapter of any one of examples 1-4, wherein the adapter volume includes a second adapter coupling feature that is removably connectable to the applicator tip at the distal adapter surface.

6. The fluid control adapter of example 5, wherein the second adapter coupling feature is also removably connectable to a second fluid control adapter positionable between the applicator tip and distal adapter surface.

7. The fluid control adapter of example 5, wherein the second adapter coupling feature is selected from tongue or groove connector, a snap, a screw, a clamp, a pin or pin-receiving opening, a loop or hook fastener, a compression fitting, a permanent magnet, an electromagnet, a temporary adhesive, a temporary bond, an electrostatic element, a click fastener, a spring fastener, or a slide fastener, wherein the second adapter coupling feature is adapted to connect with applicator tip coupling feature.

8. The fluid control adapter of any one of examples 1-7, wherein the array of proximal adapter openings, the array of distal adapter openings, or both are arranged in rows and columns, shaped patterns, staggered patterns, or random patterns.

9. The fluid control adapter of any one of examples 1-8, wherein one or more of the proximal adapter openings are large enough to span multiple distal body openings of multiple body microfluidic channels of the fluid directing body.

10. The fluid control adapter of any one of examples 1-9, wherein the adapter volume is softer at the proximal adapter surface than at the distal adapter surface.

11. The fluid control adapter of any one of examples 1-10, wherein the adapter volume is harder at an interior location compared to the proximal adapter surface, the distal adapter surface, or both.

12. The fluid control adapter of any one of examples 1-11, including an electrical, optical, thermal, or mechanical connection feature to interact when fluidly connected with the fluid directing body.

13. The fluid control adapter of any one of examples 1-12, wherein the fluid modification architecture or component includes mechanical architecture to introduce fluid passing the fluid control adapter to fluid dividing, fluid combining, fluid mixing, fluid blocking, fluid redirecting, fluid sampling, fluid trapping, air bubble separation, or a combination thereof.

14. The fluid control adapter of any one of examples 1-13, wherein the fluid modification architecture or component includes an electrical component to introduce fluid passing the fluid control adapter to electrical current, capacitance, electrophoresis, dielectrophoresis, electroshock, electrospray, electromagnetic frequency, electromagnetic field, resistance, induction, piezoelectric interaction, or a combination thereof.

15. The fluid control adapter of any one of examples 1-14, wherein the fluid modification architecture or component includes a valve.

16. The fluid control adapter of any one of examples 1-15, wherein the fluid modification architecture or component includes a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof.

17. The fluid control adapter of any one of examples 1-6, wherein the fluid modification architecture or component includes a dry reagent or a fluid reagent to interact with fluid passing the fluid control adapter.

18. The fluid control adapter of any one of examples 1-17, wherein the fluid modification architecture or component includes a thermocontroller.

19. The fluid control adapter of example 1, wherein the fluid modification. architecture or component includes a particle or cellular capture or sorting apparatus.

20. A modular fluid control adapter system, comprising:
a first fluid control adapter, comprising a first adapter microfluidic channel fluidically connecting one or more proximal adapter openings defined by a first proximal adapter surface with one or more distal adapter openings defined by a first distal adapter surface; and
a second fluid control adapter, comprising a second adapter microfluidic channel fluidically connecting one or more proximal adapter openings defined by a second proximal adapter surface with one or more distal adapter openings defined by a second distal adapter surface, wherein the first distal adapter surface is releasably sealable against the second proximal adapter surface to allow the flow of fluid from within the first adapter microfluidic channel to within the second adapter microfluidic channel,
wherein the first adapter microfluidic channel interacts mechanically, optically, chemically, biologically, or thermally with fluid when introduced therein in a manner that is different than how the second adapter interacts with the fluid.

21. The modular fluid control adapter system of example 20, wherein the second fluid control adapter is an applicator tip to apply fluid to an application surface.

22. The modular fluid control adapter system of any one of examples 20-21, wherein the first adapter microfluidic channel, the second adapter microfluidic channel, or both includes a fluid modification architecture or component.

23. The modular fluid control adapter system of example 22, wherein the fluid modification architecture or component includes a mechanical architecture to introduce fluid contained within or passing through the first fluid control adapter, the second fluid control adapter, or both to fluid dividing, fluid combining, fluid mixing, fluid sampling, fluid trapping, air bubble separation, or a combination thereof.

24. The modular fluid control adapter system of example 22, wherein the fluid modification architecture or component includes an electrical component to introduce fluid contained within or passing through the first fluid control adapter, the second fluid control adapter, or both to electrical current, capacitance, electrophoresis, dielectrophoresis, electroshock, electrospray, electromagnetic frequency, electromagnetic field, resistance, induction, piezoelectric interaction, or a combination thereof.

25. The modular fluid control adapter system of example 24, wherein the electrical component includes a resistor to generate heat, gas bubbles for fluid movement, modify voltage, reduce current, or a combination thereof.

26. The modular fluid control adapter system of example 22, wherein the fluid modification architecture or component includes a valve.

27. The modular fluid control adapter system of example 26, wherein the valve is a mechanical valve, a bubble valve, an osmotic valve, a wax valve, or a rheologic valve.

28. The modular fluid control adapter system of example 22, wherein the fluid modification architecture or component includes a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof.

29. The modular fluid control adapter system of example 22, wherein the fluid modification architecture or component includes a dry reagent or a fluid reagent to interact with fluid contained within or passing through the first fluid control adapter, the second fluid control adapter, or both.

30. The modular fluid control adapter system of example 22, wherein the fluid modification architecture or component includes a thermocontroller.

31. The modular fluid control adapter system of any one of examples 20-30, wherein the first fluid control adapter is connected to the second fluid control adapter as a fluid control adapter assembly.

32. A modular microfluidic flow cell array, comprising:
a fluid directing body defining a plurality of body microfluidic channels fluidically connecting source fluid openings with distal body openings at a distal body surface;
an applicator tip including:
   a proximal tip surface defining proximal tip openings,
   a distal tip surface defining distal tip openings fluidically connected to the proximal tip openings, the distal tip openings arranged to provide fluid communication between the applicator tip and an application surface when the distal tip surface is contacted therewith, and
   a tip coupling feature at or adjacent to the proximal tip surface, wherein the tip coupling feature is removably connectable to the fluid directing body to provide fluidic communication between the distal body openings and the proximal tip openings.

33. The modular microfluidic flow cell array of example 32, further comprising a second applicator tip that is exchangeable with the applicator tip, wherein the second applicator tip includes:
a second proximal tip surface defining second proximal tip openings,
a second distal tip surface defining second distal tip openings fluidically connected to the second proximal tip openings, the second distal tip openings arranged to provide fluid communication between the second applicator tip and the application surface when the second distal tip surface is contacted therewith, and
a second tip coupling feature at or adjacent to the second proximal tip surface, wherein the second tip coupling feature is removably connectable to the fluid directing body to provide fluidic communication between the distal body openings and the second proximal tip openings.

34. The modular microfluidic flow cell array of example 33, wherein the second applicator tip is the same as the applicator tip, providing a replacement for the applicator tip.

35. The modular microfluidic flow cell array of example 33, wherein the second applicator tip is different than the applicator tip.

36. The modular microfluidic flow cell array of example 35, wherein the second applicator tip is adapted to generate a different pattern compared to the applicator tip.

37. The modular microfluidic flow cell array of example 35, wherein the first and second applicator tips are adapted to generate substance spots, and the second applicator tip is adapted to generate a different number of substance spots compared to the applicator tip.

38. The modular microfluidic flow cell array of example 35, wherein the first and second applicator tips are adapted to generate substance spots, and the second applicator tip is adapted to generate different substance spot shapes, different substance spot sizes, or different substance spot orientations compared to the applicator tip.

39. The modular microfluidic flow cell array of example 35, wherein the second applicator tip includes a different material that is harder or softer compared to the applicator tip.

40. The modular microfluidic flow cell array of example 35, wherein the second applicator tip has a different thickness compared to the applicator tip.

41. The modular microfluidic flow cell array of any one of examples 32-40, wherein the applicator tip, the second applicator tip, or both includes a fluid modification architecture or component that interacts mechanically, optically, chemically, biologically, or thermally with fluid during at least a portion of the time that fluid is contained within or passing through the applicator tip or the second applicator tip.

42. The modular microfluidic flow cell array of example 41, wherein the fluid modification architecture or component includes a mechanical architecture to introduce fluid contained within or passing through the first applicator tip or the second applicator tip when fluidically connected to fluid dividing, fluid combining, fluid mixing, fluid sampling, fluid trapping, air bubble separation, or a combination thereof.

43. The modular microfluidic flow cell array of example 41, wherein the fluid modification architecture or component includes an electrical component to introduce fluid contained within or passing through the first applicator tip or the second applicator tip when connected to electrical current, capacitance, electrophoresis, dielectrophoresis, electroshock, electrospray, electromagnetic frequency, electromagnetic field, resistance, induction, piezoelectric interaction, or a combination thereof.

44. The modular microfluidic flow cell array of example 43, wherein the electrical component includes a resistor to generate heat, generate or manipulate gas bubbles, modify voltage, reduce current, or a combination thereof.

45. The modular microfluidic flow cell array of example 41, wherein the fluid modification architecture or component includes a valve.

46. The modular fluid control adapter system of example 45, wherein the valve is a mechanical valve, a bubble valve, an osmotic valve, a wax valve, or a rheologic valve.

47. The modular microfluidic flow cell array of example 41, wherein the fluid modification architecture or component includes a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof.

48. The modular microfluidic flow cell array of example 41, wherein the fluid modification architecture or component includes a dry reagent or a fluid reagent to interact with fluid contained within or passing through the first application tip or the second application tip when connected.

49. The modular microfluidic flow cell array of example 41, wherein the fluid modification architecture or component includes a thermocontroller.

50. The modular microfluidic flow cell array of any one of examples 32-49, further comprising a fluid control adapter adapted to be positioned between the directing body and the applicator tip.

51. The microfluidic flow cell array of any one of examples 32-50, wherein the applicator tip is integrated with an application surface.

52. The microfluidic flow cell array of any one of examples 32-51, wherein the applicator tip and the second applicator tip are integrated with an application surface at different locations along the application surface.

53. A modular microfluidic flow cell array, comprising:
a fluid directing body defining a plurality of body microfluidic channels fluidically connecting source fluid openings with distal body openings at a distal body surface;
a first fluid control adapter including a first adapter microfluidic channel fluidically connecting one or more proximal adapter openings defined by a first proximal adapter surface with one or more distal adapter openings defined by a first distal adapter surface, wherein the first proximal adapter surface is removably connectable with the distal body surface of the fluid directing body; and
an applicator tip removably connectable with the first distal adapter surface of the first fluid control adapter,
wherein when the fluid directing body, the first fluid control adapter, and the applicator tip are fluidically connected, the substance applied to an application surface occurs as fluid passes from the fluid directing body, through the first fluid control adapter, and through the applicator tip depositing the substance to the application surface.

54. The modular microfluidic flow cell array of example 53, wherein the first adapter microfluidic channel interacts mechanically, optically, chemically, biologically, or thermally with fluid during at least a portion of the time that fluid is contained within or passing through the first fluid control adapter.

55. The modular microfluidic flow cell array of any one of examples 53-54, wherein the first adapter microfluidic channel includes mechanical architecture to introduce fluid contained within or passing through the first fluid control adapter to fluid dividing, fluid combining, fluid mixing, fluid sampling, fluid trapping, air bubble separation, or a combination thereof.

56. The modular microfluidic flow cell array of any one of examples 53-55, wherein the first adapter microfluidic channel includes an electrical component to introduce fluid contained within or passing through the first fluid control adapter to electrical current, capacitance, electrophoresis, dielectrophoresis, electroshock, electrospray, electromagnetic frequency, electromagnetic field, resistance, induction, piezoelectric interaction, or a combination thereof.

57. The modular microfluidic flow cell array of any one of examples 53-56, wherein the first adapter microfluidic channel includes a valve.

58. The modular microfluidic flow cell array of any one of examples 53-57, wherein the first adapter microfluidic channel includes a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof.

59. The modular microfluidic flow cell array of any one of examples 53-58, wherein the first adapter microfluidic channel carries a dry reagent or a fluid reagent to interact with fluid contained within or passing through the first fluid control adapter.

60. The modular microfluidic flow cell array of example 53, wherein the first adapter microfluidic channel includes a thermocontroller.

61. The modular microfluidic flow cell array of any one of examples 53-60, further comprising a second fluid control adapter having a second adapter microfluidic channel fluidically connecting one or more proximal adapter openings defined by a second proximal adapter surface with one or more distal adapter openings defined by a second distal adapter surface, wherein the second fluid control adapter is positionable between the first fluid control adapter and the applicator tip or is interchangeable with the first fluid control adapter.

62. The modular microfluidic flow cell array of example 61, wherein the second fluid control adapter is positioned between the first fluid control adapter and the applicator tip, wherein the second adapter microfluidic channel fluidically connects the first adapter microfluidic channel with an applicator flow channel of the applicator tip.

63. The modular microfluidic flow cell array of example 61, wherein the first adapter microfluidic channel interacts mechanically, optically, chemically, biologically, or thermally with fluid during at least a portion of the time that fluid is contained within or passing through the first fluid control adapter, wherein the second adapter microfluidic channel also interacts mechanically, optically, chemically, biologically, or thermally with fluid during at least a portion of the time that fluid is contained within or passing through the first fluid control adapter, and wherein the first adapter microfluidic channel and the second adapter microfluidic channel interact differently with the fluid relative to one another.

64. The modular microfluidic flow cell array of any one of examples 53-63, wherein the applicator tip is integrated with the application surface.

65. The modular fluid control adapter system of example 64, wherein a second applicator tip is also integrated with the application surface at a different location along the application surface relative to the applicator tip, wherein the second applicator tip is also removably attachable to the first distal adapter surface of the first fluid control adapter.

66. The modular fluid control adapter system of example 64, wherein the distal body surface is sealed with the first proximal adapter surface and the first distal adapter surface is sealed with the a distal tip surface of the applicator tip, and wherein the fluid directing body, the first fluid control adapter, and the applicator tip are fluidically connected together to permit fluid to pass from within the fluid directing body and through the applicator tip.

67. The modular fluid control adapter system of example 64, wherein the first fluid control adapter includes flexible hollow conduit having flow channels longer than a width or a depth of the first fluid control adapter.

68. The modular fluid control adapter system of example 64, wherein the first fluid control adapter is configured to preserve the microfluidic connection to the applicator tip integrated with the application surface even when the fluid directing body is moved in an x-direction, a y-direction, a z-direction, or a combination of directions thereof.

69. The modular fluid control adapter system of any one of examples 53-68, wherein the connection and disconnection of the fluid directing body, the first fluid control adapter, and the applicator tip occurs by automation.

70. The modular fluid control adapter system of any one of examples 53-69, wherein the exchange of the first fluid control adapter with a second fluid control adapter, or adding the second fluid control adapter between the first fluid control adapter and the applicator tip occurs by automation.

71. The modular fluid control adapter system of any one of examples 53-70, wherein the connection between the distal body surface of the fluid directing body and the proximal adapter surface of the first fluid control adapter also makes electrical, optical, thermal, or mechanical connection.

72. The modular fluid control adapter system of any one of examples 53-71, wherein the connection between the first distal adapter surface of the first fluid control adapter also makes electrical, optical, thermal, or mechanical connection with the applicator tip or a second fluid control adapter positioned between the first fluid control adapter and the applicator tip.

73. The modular fluid control adapter system of any one of examples 53-72, wherein the first fluid control adapter includes multiple first adapter microfluidic channels.

74. The modular fluid control adapter system of any one of examples 53-73, wherein the first fluid control adapter includes one or more proximal adapter openings associated with unconnected channels that do not permit the fluid to pass into the applicator tip.

75. The modular fluid control adapter system of any one of examples 53-74, wherein when the fluid directing body, the first fluid control adapter, and the applicator tip are fluidically connected and the substance has been deposited on the application surface, a return flow path is present to return the fluid through the first fluid control adapter and into the fluid directing body.

76. A method of treating an application surface for analysis of a substance, comprising:
docking a first modular microfluidic flow cell array on an application surface, wherein the first modular microfluidic flow cell array includes a fluid directing body fluidly connected to a first fluid control adapter;
flowing a first fluid through the first modular microfluidic flow cell array including through the first fluid control adapter;
removing the first fluid control adapter from the fluid directing body;
fluidly connecting a second fluid control adapter to the fluid directing body to form a second modular microfluidic flow cell array;
docking the second modular microfluidic flow cell array on the application surface; and
flowing a second fluid through the second modular microfluidic flow cell array including through the second fluid control adapter.

77. The method of example 76, wherein the first fluid and the second fluid are the same.

78. The method of any one of examples 76-77, wherein the first fluid and the second fluid are different.

79. The method of any one of examples 76-78, wherein the first fluid control adapter interacts differently with the fluid compared to the second fluid control adapter.

80. The method of any one of examples 76-79, wherein the first fluid control adapter, the second fluid control adapter, or both are in the form of a first applicator tip, a second applicator tip, or both, respectively.

81. The method of any one of examples 76-80, wherein the first fluid control adapter, the second fluid control adapter, or both are not in the form of applicator tips, and the method further includes:
fluidly connecting a first applicator tip to the first fluid control adapter to flow the first fluid also through the first applicator tip to contact the application surface via the first applicator tip;
fluidly connecting a second applicator tip to the second fluid control adapter to flow the second fluid also through the second applicator tip to contact the application surface via the second applicator tip; or
both.

82. The method of any one of examples 76-81, wherein flowing the first fluid, flowing the second fluid, or both includes flowing fluid for a purpose other than depositing substance spots on the application surface.

83. The method of example 82, wherein flowing the first fluid or flowing the second fluid is not for application of substance spots, but rather is for surface preparation, deposition spot washing, microfluidic channel priming, generating a reaction, removing materials, swapping materials, manipulating cells, or a combination thereof.

84. The method of any one of examples 76-83, wherein flowing the first fluid results in depositing a first group of substance spots on the application surface, flowing the second fluid results in depositing a second group of substance spots on the application surface, or both.

85. The method of example 84, further comprising depositing the first group of substance spots and the second group of substances spots at overlapping interstitial locations on the application surface.

86. The method of example 85, wherein the overlapping interstitial locations are partially overlapping with one or both of the first group of substance spots or the second group of substance spots being applied to space on the application surface without the first group of substance spots.

87. The method of example 85, wherein the overlapping interstitial locations are fully overlapping.

88. The method of example 84, further comprising depositing the first group of substance spots and the second group of substances spots at non-overlapping interstitial locations on the application surface.

89. The method of example 84, wherein unspotted areas or uniformly spotted areas of the first or second group of substance spots are used for internal referencing.

90. The method of example 84, wherein the first modular microfluidic flow cell array and the second modular microfluidic flow cell array are arranged to deposit different patterns of substance on the application surface.

91. The method of example 84, further comprising applying an interrogation fluid to a plurality of spots or all spots of the first group of substance spots, the second group of substances spots, or both.

92. The method of example 91, wherein the interrogation fluid is also applied on unspotted space on the application surface surrounding the plurality of spots or all of the spots of the first group of substance spots, the second group of substances spots, or both.

93. The method of example 91, wherein the interrogation fluid is applied using an interrogation applicator tip that is fluidically connectable directly or indirectly to the fluid directing body.

94. The method of example 91, wherein the interrogation applicator tip is fluidically connectable and disconnectable by mechanical, magnetic, or electrostatic force engagement and disengagement.

95. The method of example 84, wherein depositing the first group of substance spots, the second group of substances spots, or both includes associating location data with the first group of substance spots, the second group of substance spots, or both by engaging a location sensor identifier with the application surface, the first modular microfluidic flow cell array, the second modular microfluidic flow cell array, the first group of substance spots, the second group of substance spots, or a combination thereof.

96. The method of any one of examples 76-95, wherein removing the first fluid control adapter from the fluid directing body and fluidly connecting a second fluid control adapter to the fluid directing body is carried out by mechanical disengagement of the fluid directing body with the first fluid control adapter and mechanical engagement of the fluid directing body with the second fluid control adapter.

97. The method of any one of examples 76-96, wherein removing the first fluid control adapter from the fluid directing body and fluidly connecting a second fluid control adapter to the fluid directing body is carried out by magnetic disengagement of the fluid directing body with the first fluid control adapter and magnetic engagement of the fluid directing body with the second fluid control adapter.

98. The method of any one of examples 76-97, wherein removing the first fluid control adapter from the fluid directing body and fluidly connecting a second fluid control adapter to the fluid directing body is carried out by electrostatic force disengagement of the fluid directing body with the first fluid control adapter and electrostatic force engagement of the fluid directing body with the second fluid control adapter.

99. The method of any one of examples 76-98, wherein removing the first applicator tip from the first fluid control adapter and fluidly connecting the second applicator tip to the second fluid control adapter is carried out by mechanical disengagement of the first fluid control adapter with the first applicator tip and mechanical engagement of the second fluid control adapter with the second applicator tip.

100. The method of any one of examples 76-99, wherein removing the first applicator tip from the first fluid control adapter and fluidly connecting the second applicator tip to the second fluid control adapter is carried out by magnetic disengagement of the first fluid control adapter with the first applicator tip and magnetic engagement of the second fluid control adapter with the second applicator tip.

101. The method of any one of examples 76-100, wherein removing the first applicator tip from the first fluid control adapter and fluidly connecting the second applicator tip to the second fluid control adapter is carried out by electrostatic force disengagement of the first fluid control adapter with the first applicator tip and electrostatic force engagement of the second fluid control adapter with the second applicator tip.

102. The method of any one of examples 76-101, wherein the first modular microfluidic flow cell array, the second modular microfluidic flow cell array, or both include data collection hardware or indicia to collect data related to the first fluid, the second fluid, or both; contacting the application surface, depositing substance spots on the application surface, or both.

103. The method of example 102, wherein the data collection hardware or indicia includes an RFID tag, a 2D barcode, an electrical contact, or optics, wherein the data collection hardware is associated with a processor or processors associated with the first modular microfluidic flow cell array, the second modular microfluidic flow cell array, the application surface, or a combination thereof.

104. The method of example 102, wherein the processor or processors are configured to convey adjustments to an operation selected from changing fluid flow parameters, expanding or restricting ports, contact pressure with the application substrate, temperature adjustment, valve operation, activating sensors, electromechanical manipulations, acoustic manipulations, optical manipulations, or a combination thereof.

105. The method of example 104, wherein the processor or processors are in the form of an internal chip or chips.

106. The method of example 104, wherein the internal chip or chips are located on the first modular microfluidic flow cell array, the second modular microfluidic flow cell array, or both at a location or locations that contacts the application surface when flowing the first fluid, flowing the second fluid, or both occurs, leaving a first group of substance spots, a second group of substance spots, or both.

107. The method of example 106, wherein the internal chip or chips include an interlocking feature creating a fluid-tight seal with the application surface or withstanding pulling forces associated with release from the application surface.

108. The method of any one of examples 76-107, wherein the fluid directing body, the first fluid control adapter, the second fluid control adaptor, or a combination thereof includes chamfers or guidance indicators to enhance fluidically coupling when engaging the fluid directing body with the first fluid control adapter, the second fluid control adaptor, or both.

109. The method of any one of examples 76-108, further comprising thermally interacting with fluid contained within or passing through the first fluid control adapter, the second fluid control adapter, or both.

110. The method of any one of examples 76-109, further comprising mechanically interacting with fluid contained within or passing through the first fluid control adapter, the second fluid control adapter, or both by fluid dividing, fluid combining, fluid mixing, fluid sampling, fluid trapping, air bubble separation, or a combination thereof.

111. The method of any one of examples 76-110, further comprising electrically interacting with fluid contained within or passing through the first fluid control adapter, the second fluid control adapter, or both using electrical current, capacitance, electrophoresis, dielectrophoresis, electroshock, electrospray, electromagnetic frequency, electromagnetic field, resistance, induction, piezoelectric interaction, or a combination thereof.

112. The method of any one of examples 76-111, further comprising sensing a fluid or substance property contained within or passing through the first fluid control adapter, the second fluid control adapter, or both using an optical sensor, a chemical sensor, a microelectromechanical system sensor, an electrical sensor, a biological sensor, an NMR sensor, a dielectrophoretic sensor, a capacitance sensor, or a combination thereof.

113. The method of any one of examples 76-112, further comprising chemically interacting with fluid contained within or passing through the first fluid control adapter, the second fluid control adapter, or both, using the first adapter microfluidic channel by contacting the fluid with a dry reagent or a fluid reagent contained therein.

114. The method of any one of examples 76-113, wherein removing the first fluid control adapter from the fluid directing body and fluidly connecting a second fluid control adapter to the fluid directing body occurs by automation.

115. The method of any one of examples 76-114, wherein:
flow of the first fluid while the first modular microfluidic flow cell array is sealed against the application surface further includes return of the first fluid through the first fluid control adapter and into or through the fluid directing body;
flow of the second fluid while the second modular microfluidic flow cell array is sealed against the application surface further includes return of the second fluid through the second fluid control adapter and into or through the fluid directing body; or
both.

116. The method of any one of examples 76-115, further comprising undocking the first modular microfluidic flow cell array from the application surface after flowing the first fluid through the first modular microfluidic flow cell array.

117. A method of applying substances for analysis to an application surface, comprising:
fluidly connecting a fluid directing body with a first applicator tip to form a first microfluidic flow cell array;
docking the applicator tip of the first modular microfluidic flow cell array on an application surface;
flowing a first fluid through the fluid directing body and the first applicator tip; disconnecting the fluid directing body from the first applicator tip;
fluidly connecting the fluid directing body with a second applicator tip to form a second microfluidic flow cell array;
docking the second applicator tip of the second modular microfluidic flow cell array on the application surface;
flowing a second fluid through the fluid directing body and the second applicator tip,
wherein flowing the first fluid and flowing the second fluid results in application of at least one set of substance spots on the application surface.

118. The method of example 117, wherein flowing the first fluid and flowing the second fluid results in application of a first group of substance spots and a second group of substance spots, respectively, on the application surface.

119. The method of example 118, wherein:
while depositing the first group of substance spots, flowing at least a portion of the first fluid away from the application surface through the first applicator tip and into or through the fluid directing body;
while depositing the second group of substance spots, flowing at least a portion of the second fluid away from the application surface through the second applicator tip and into or through the fluid directing body; or
both.

120. The method of example 119, wherein the first applicator tip, the second applicator tip, or both, include flexible hollow conduit.

121. The method of example 120, wherein the first applicator tip, the second applicator tip, or both are configured to preserve the microfluidic connection to the fluid directing body even when the fluid directing body or the application surface is moved in an x-direction, a y-direction, a z-direction, or a combination of directions thereof.

122. The method of any one of examples 117-121, further comprising:
fluidically connecting a first fluid control adapter between the fluid directing body and the first applicator tip;
fluidically connecting a second fluid control adapter between the fluid directing body and the second applicator tip; or
both.

123. The method of example 122, wherein the first fluid control adapter, the second fluid control adapter, or both include flexible hollow conduit.

124. The method of example 123, wherein the first fluid control adapter, the second fluid control adapter, or both, are configured to preserve the microfluidic connection to the first applicator tip, the second applicator tip, or both respectively even when the fluid directing body or the application surface is moved in an x-direction, a y-direction, a z-direction, or a combination of directions thereof.

125. The method of any one of examples 117-124, wherein flowing the first fluid or flowing the second fluid is not for application of substance spots, but rather is for surface preparation, deposition spot washing, microfluidic channel priming, generating a reaction, removing materials, swapping materials, manipulating cells, or a combination thereof.

126. The method of any one of examples 117-125, wherein the first applicator tip, the second applicator tip, or both are impermanently joined with the application surface, but are joined sufficiently to withstand pulling pressure applied when disconnecting from the fluid directing body.

127. The method of any one of examples 117-126, wherein the first applicator tip, the second applicator tip, or both, and the application surface are in the form of a consumable.

128. The method of any one of examples 117-127, further comprising undocking the first modular microfluidic flow cell array from the application surface after flowing the first fluid through the first modular microfluidic flow cell array.

### Definitions

In describing embodiments of the present disclosure, the following terminology will be used. Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

The singular forms “a,” “an” and “the” include plural referents unless the context clearly dictates otherwise. Thus, for example, reference to “a flow cell includes reference to one or more flow cells.

As used herein, “comprising” or “including” language or other open-ended language can be substituted with “consisting essentially of” and “consisting of” as if such transition phrase is expressly included in such embodiments.

A “modular microfluidic flow cell array” (MMFCA) is defined herein to include a fluid directing body and one or more fluid control adapters attached or attachable to the fluid directing body in the form of an assembly or a kit for assembly. A fluid control adapter may be in the form of an applicator tip that includes one or more fluid modification architecture(s). Alternatively, one or more fluid control adapters can be fluidically coupled with an applicator tip that does not include a fluid modification architecture. The modular aspect of these modular microfluidic flow cell arrays is provided by the fluid control adapter being attachable and removable from the fluid directing body for exchange with another fluid control adapter. In some instances, the fluid control adapter can be stackable with other fluid control adapters to provide additional functionality. When the fluid directing body is fluidly connected to one or more fluid control adapters, a flow cell can be formed for depositing a substance of a fluid onto an application surface. In some instances, the fluid control adapter can be in the form of an applicator tip that is sealed against the application surface for substance application and/or in some instances the fluid control adapter can be an intermediate fluid control adapter positioned between the fluid directing body and the applicator tip. “Fluid modification architecture or component” refers to any mechanical, optical, physical, chemical, electrical, etc., device, compound, or composition that interacts with a fluid within a modular microfluidic flow cell array (typically within the fluid control adapter and/or the applicator tip) in a manner that senses or modifies a flow characteristic of a flow cell, or in some cases, may even modify the physical and/or chemical properties of the fluid flowing through the modular microfluidic flow cell arrays and/or systems of the present disclosure. Examples may include architecture(s) and/or component(s) that provide fluid combining, fluid dividing, fluid redirecting for a functional purpose, electrical interaction, valving, thermocontrolling, fluid sensing, reagent mixing or pickup, fluid mixing or agitation, fluid storage, fluid blocking or fluid return (without reaching the application surface), etc. The fluid modification architecture(s) or component(s) is defined to include any architecture and/or component that is other than the exterior walls that laterally circumscribe the cross-sectional general shape of any of the microfluidic channels (orthogonal to fluid flow) of the fluid control adapter(s) and/or applicator tip(s). For example, a round or a square cross-sectional shaped microfluidic channel would not be considered to be a fluid modification architecture or component, but a round or square cross-sectional shaped microfluidic channel modified with a protrusion for mixing, a coating for modification of wetting properties, a reagent contained within a microfluidic channel, a fluid divider or combiner configuration to separate or join, respectively, fluid flow paths, etc., would be considered to be a fluid modification architecture or component.

A “flow cell” of a modular microfluidic flow cell array is defined herein to include a flow path for channeling fluid that is formed when at least a fluid directing body is fluidically coupled with a fluid control adapter. The fluid control adapter may be in the form of an applicator tip, or may be configured to connect to a separate applicator tip (or other intervening fluid control adapters followed by an applicator tip). A flow cell also includes a flow chamber as part of an applicator tip (which could also itself be a fluid control adapter) and is positioned to interface with an application surface for substance deposition. The flow path formed within an assembled modular microfluidic flow cell array includes fluidly connected microfluidic channels from these various assembled structures, thereby forming one or more flow cells. The flow path provides ingress of fluid to the flow chamber for substance deposition onto a deposition surface. In some examples, a second microfluidic flow path provides egress from the flow chamber. In this latter example, fluid can be flowed bi-directionally (back and forth) through the flow paths into and out of the flow chamber of the applicator tip. Thus, the fluid contained within or passing through the flow chamber may deposit a substance carried by the fluid on the application surface to which the flow chamber is sealed. The modular microfluidic flow cell array of the present disclosure allows the user to design various flow cell arrangements by selecting from an assortment of fluid control adapters and/or stacking (combining) fluid control for a variety of substance deposition functionalities. Multiple flow cells of a modular microfluidic flow cell array can generate any number of substance spots on an application surface via its flow chamber, depending on design, with a few examples including an array of flow chambers at 4×4, 5×5, 6×6, 8×8, 4×8, 1×8, 2×16, 4×24, 8×12, 6×8, 6×16, 8×24, and any other arrangement or any shape of flow chambers that can be fit at a distal tip surface of an applicator tip. The number of flow cells with corresponding flow chambers at a distal tip surface of an applicator tip can be, for example, up to 48, 96, 192, 384, 768, or more, e.g., from 2 to 768 flow cells, from 4 to 192 flow cells, from 8 to 96 flow cells, etc.

A “flow chamber” of a flow cell is typically in the form of an open cavity defined at its distal tip surface which can form a contact deposition seal on an application surface. A first tip microfluidic channel provides fluid ingress to the flow chamber, and in some examples a second tip microfluidic channel provide egress from the flow chamber. Ingress and egress may be swapped, such as for bi-directional flow applications. The distal tip surface that forms the contact deposition seal surrounding the open cavity may be pressed against the application surface for any of a number of fluid flow purposes. In some instances, the flow chamber may act as a deposition chamber, where substances passing therethrough are deposited on the application surface. In other examples the flow channel may act as a washing chamber or a substance removal chamber, and thus, is not always used for substance application to the application surface. In each use scenario, however, fluid is flowed into and out of the flow chamber, and thus, the term “flow chamber” adequately describes each of these various types of uses. Even in instances where the fluid might temporarily be held stagnant within the flow chamber against the application surface, e.g., soaking the application surface or simply pausing between flows, there would still be fluid flow before or after such a fluid hold within the flow chamber.

“Microfluidic channel(s)” is a term that may be interchangeable with terms such as channels, microchannels, conduits, microconduits, canals, microcanals, tubules, microtubules, tubes, microtubes, or the like. Microfluidic channels can be defined within any of a number of structures in the present disclosure, such as fluid directing bodies, fluid control adapters, applicator tips, etc., which can be connected together modularly to form a flow path of a microfluidic flow cell or multiple flow paths of an array of microfluidic flow cell arrays. To distinguish which portion of the microfluidic channel is carried by which modular portion of the various structures, the terms “body microfluidic channel(s),” “adapter microfluidic channel(s),” and “tip microfluidic channel(s)” is used herein. The connection of any of these structures together forms a “microfluidic flow path” or “flow path” that typically spans multiple components of a modular microfluidic flow cell array when assembled. Furthermore, the microfluidic channels in general may be formed on the micro-scale, providing microfluidic pathways (of any cross-sectional shape) which are typically used to guide the substance(s) to and from an application surface via their independently fluidly coupled flow chamber(s), often providing a fluid flow that produces a high surface concentration of a substance at a specific region (or spot) on an application surface. Each deposition region can be individually addressed with its own flow cell, and a (modular) microfluidic flow cell array may be arranged such that a large number of deposition regions may be addressed in parallel.

When referring to the “cross-sectional” shape, size, dimension, area, etc., of a microfluidic channel(s), it is understood that the term “cross-sectional” in this context refers to the plane of the microfluidic channel that is perpendicular to the direction of fluid flow.

A “large flow cell applicator,” “LFC,” or “single large flow cell applicator” is similar to a microfluidic flow cell array, but in accordance with the present disclosure, an LFC has a single flow cell (not multiple flow cells) and is often used to overprint (or over spot) or underprint (or underspot) on an application surface at the same location as multiple spots applied by a microfluidic flow cell array. The term “large” simply indicates that the flow chamber size of the flow cell is large enough to apply fluid at the same location as a plurality of spots applied using a microfluidic flow cell array. However, it is noted that in accordance with examples of the present disclosure, a fluid control adapter can be configured to provide the function of a large flow cell, thus giving the ability or option to provide a modular fluid control adapter system or large flow cell array system that does not have a dedicated large flow cell applicator.

The term “application surface” refers to any surface or combination of surfaces to which a distal tip surface (forming a contact deposition seal) of a modular microfluidic flow cell array is pressed to receive a fluid, a fluid substance, or a fluid carrying a substance. The application surface may be an inert substrate that does not interact with the fluid or substance, or the application surface may interact with the fluid or substance. In some instances, the application surface may be a surface of a coating applied to a substrate, or the application surface may be provided solely by a single substrate, for example. The application surface may be an assay surface in some examples. It is noted that the term “application” does not indicate that every interaction between a fluid and the application surface results in the application of a substance spot, but rather, can include fluid interactions with the application surface that do not leave a substance behind for evaluation, e.g., application surface preparation, deposition spot washing, microfluidic channel priming, generating a reaction, removing materials, swapping materials, manipulating cells, or a combination thereof.

The term “assay surface” refers to a surface of a sensor chip, e.g., thin metal film, metal coupon, grating, etc., of a sensing substrate, e.g., sensor chip with or without a solid optical material, where material may be spotted with a flow cell array and where chemical interactions can occur. In the context of SPR, the assay surface can be one side of a sensor chip that may include supplemental coatings, pre-applied ligands, etc., and can be spotted or applied for generating substance interactions. Thus, the assay surface may be transparent, translucent, thermally conductive, electrically conductive, insulated, etc. Sensors associated with the assay surface may be mechanical, optical, physical, chemical, electrical, or the like, and can detect mechanical properties, e.g., pressure or flow, temperature, optical properties, e.g., SPR, electrical properties, e.g., capacitance or conductivity, chemical properties, such as pH or colorimetric analysis, etc.

The term “solid optical material” refers to any solid shape of optical material where light can enter and interact with a deposited sample, directly or indirectly (SPR), e.g., optical prism for beam shaping, or some other beam shaping configuration. In many instances, an optical prism will be described with some specificity. It is noted, however, that the prism can be any shape that is suitable for shaping light energy for use with the sensing substrate in accordance with examples of the present disclosure.

The term “chip” or “sensor chip” refers to a data collection component used for measuring surface interactions, typically on the application surface. A sensor chip can include a thin layer(s) of material (such as a metal film or a film of another material, for example) applied to a solid optical material, a grating structure, or a coupon of material (such as metal or other material, for example). The sensor chip does not include the solid optical material but may be applied to the solid optical material. The sensor chip can include multiple surfaces, including an assay surface where chemical or other interactions can occur, and an optical surface where optics can sense the interactions. Specifically, in an SPR configuration and some other similar types of sensor configurations, the assay surface can be positioned opposite the optical interface surface. In other configurations, however, the assay surface and the optical interface surface can be the same surface, or there can be a different spatial relationship between these two surfaces. Regardless, the “sensor chip” may be preloaded with a supplemental coating(s), ligand(s), or any other material(s) that may be useful for evaluating substance interactions. The sensor chip may be referred to as a “thin metal layer” or “thin layer” in some more specific examples, or as a “metal coupon” or “coupon” in other specific examples. It is noted that in some examples, the sensor chip may be affixed or attached to the solid optical material (as a film or adhered thereto) or can be set in place in contact with a facial surface of the solid optical material (as a sensing substrate that is modular), for example. For clarity, in another context, the term “chip” can alternatively refer to a component that uses memory for storing data, and thus can be referred to more specifically as a “data chip,” “memory chip,” or the like.

The term “SPR” or “surface plasmon resonance” refers to optical sensing systems that use a light energy source optically coupled to an application surface, and may include a sensor chip with an assay surface and a detector to receive reflected light from the optical interface surface.

A “substance” refers to a fluid or a material carried by a fluid that may include particles, molecules, compounds, or other species of materials that are used to conduct experiments on application surfaces, e.g., to be sensed using the optical interface surface of a (sensor) chip or by some other sensing technology. The substance can include an analyte, a particle, a probe, an immobilized ligand, etc., depending on the context.

The term “spot” refers to a sample applied at a discrete location on an application surface. The sample can be applied as a fluid sample that dries, or can remain undried. In some instances a second sample can be applied to the same location in an overlapping manner. Sometimes spots are applied by the modular microfluidic flow cell arrays described herein and then other spots or regions of the application surface may be applied adjacently or overlaid (partially or fully) with other “spots” of typically a different sample (different substance, different substance concentration in a fluid sample, different spot size, etc.).

The terms “print,” “printed,” or “printing” in the context of the present disclosure typically refers to the application of substance spot from a fluid or the flowing of a fluid across an application surface via a flow chamber in contact with the application surface. Thus, “printing” can be carried out by depositing a substance or substance spot(s) from a fluid onto an application surface, or can include the flow of fluid across an application surface for a reason other deposition of a substance or substance spot(s) onto the application surface, e.g., application surface preparation, deposition spot washing, microfluidic channel priming, generating a reaction, removing materials, swapping materials, manipulating cells, or a combination thereof. Thus, the terms “print,” “printed,” or “printing” should be interpreted broadly in to include any application or contact of a fluid with an application surface via fluid flow into or through a flow chamber, whether the purpose is to leave a substance spot behind or to carry out some other fluid interaction function. In some instances, the terms “apply,” “applied,” or “applying” may be used as well, and should be given the same broad interpretation in the context of flow chamber fluid flow.

It is noted that the terms “first,” “second,” and so forth, are used herein and throughout the present disclosure. In some instances, the term “third,” “additional” or “other” may be used to describe flow cells or other structures beyond the first and second arrangements identified. These terms are meant to be relative to one another only in the context in which they are mentioned, and further, do not infer any order of use that any one of these terms should be associated exclusively with a specific component. For example, a first flow cell could be referred to as a second flow cell or vice versa. In some instances, the “first” flow cell may be referred to as simply a “flow cell,” as the term “first” is simply used for clarity when describing the flow cell applicator relative to a second (or third, or fourth, etc.) flow cell. Thus, these may be described as a flow cell and a second flow cell in some instances, which refers to the same two structures unless the context dictates otherwise. As another example, if a first flow cell applies a first group of substance spots, and then applies a second group of substance spots, a second flow cell can apply a third group of substance spots (from a microfluidic flow cell array) or a single spot from a large flow cell (LFC) applicator, and so forth. However, in accordance with examples of the present disclosure, any of these functionalities can be accomplished by the use of a fluid control adapter configured to provide one or more of these application functions.

Numerical data (numbers of elements, amounts, dimensions, etc.) may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 μm” should be interpreted to include not only the explicitly recited values of about 50 μm and 250 μm, but also the individual values and sub-ranges within the indicated range. Thus, included in this numerical range are individual values such as 60, 70, and 80 μm, and sub-ranges such as from 50-100 μm, from 100-200 μm, and from 100-250 μm, etc. This same principle applies to ranges reciting only one numerical value and should apply regardless of the breadth of the range or the characteristics being described.

As used herein, the term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about shall expressly include “exactly, consistent with the discussion above regarding ranges and numerical data. For example, the term “about” can refer to the recited number plus or minus 5%, plus or minus 3%, or plus or minus 1%. To illustrate, the term “about” when interpreted as being plus or minus 5% of a numeric range, such as “from about 1 cm to about 2 cm,” would be interpreted as including a range from 9.5 mm to 2.1 cm, from 1.05 cm to 1.9 cm, from 9.5 mm to 1.9 cm, or 1.05 cm to 2.1 cm.

Similar calculations for any of the other individual numerical values or individual parameters of numerical ranges set forth herein can be modified similarly such that the “about” modifier fully supports subranges including +/−3% or +/−1% of the numerical value provided.

The term “example(s)” or “embodiment(s),” particularly when followed by a listing of terms or a description of a particular structure, is merely exemplary and illustrative and should not be deemed to be exclusive or comprehensive.

The devices, systems, methods, and/or compositions disclosed herein are not limited to particular methodology, protocols, reagents, etc., because, as the skilled artisan will appreciate, they may vary. Further, the terminology used herein is for the purpose of describing particular embodiments only, and is not intended to and does not limit the scope of that which is disclosed or claimed.

1. A fluid control adapter, comprising:
a proximal adapter surface including an array of proximal adapter openings to receive fluid from a fluid directing body when fluidly connected therewith;
a distal adapter surface including an array of distal adapter openings to pass fluid to an applicator tip when fluidly connected therewith;
an adapter volume defining a plurality of adapter microfluidic channels individually fluidically connecting one or more proximal adapter openings with one or more distal adapter openings; and
a fluid modification architecture or component within the adapter volume to interact with fluid contained within or passing through the one or more of the adapter microfluidic channels.

2. The fluid control adapter of claim 1, further comprising a first adapter coupling feature at the proximal adapter surface that is removably connectable to the fluid directing body.

3. The fluid control adapter of claim 2, wherein the first adapter coupling feature:
is removably connectable to a second fluid control adapter positionable between the fluid directing body and the proximal adapter surface of the fluid control adapter;
includes a feature selected from a tongue or groove connector, a snap, a screw, a clamp, a pin, a loop or hook fastener, a compression fitting, a permanent magnet, an electromagnet, a temporary adhesive, a temporary bond, an electrostatic element, a click fastener, a spring fastener, or a slide fastener, and wherein the first adapter coupling feature is adapted to connect with a body coupling feature; or
a combinations thereof

4. (canceled)

5. The fluid control adapter of claim 1, wherein the adapter volume includes a second adapter coupling feature that is removably connectable to the applicator tip at the distal adapter surface.

6-11. (canceled)

12. The fluid control adapter of claim 1, including an electrical, optical, thermal, or mechanical connection feature to interact when fluidly connected with the fluid directing body.

13. The fluid control adapter of claim 1, wherein the fluid modification architecture or component includes:
mechanical architecture to introduce fluid passing the fluid control adapter to fluid dividing, fluid combining, fluid mixing, fluid blocking, fluid redirecting, fluid sampling, fluid trapping, air bubble separation, or a combination thereof;
an electrical component to introduce fluid passing the fluid control adapter to electrical current, capacitance, electrophoresis, dielectrophoresis, electroshock, electrospray, electromagnetic frequency, electromagnetic field resistance, induction, piezoelectric interaction, or a combination thereof;
a valve;
a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof;
a dry reagent or a fluid reagent to interact with fluid passing fluid control adapter;
a thermocontroller;
a particle or cellular capture or sorting apparatus; or
a combination thereof.

14-52. (canceled)

53. A modular microfluidic flow cell array, comprising:
a fluid directing body defining a plurality of body microfluidic channels fluidically connecting source fluid openings with distal body openings at a distal body surface;
a first fluid control adapter including a first adapter microfluidic channel fluidically connecting one or more proximal adapter openings defined by a first proximal adapter surface with one or more distal adapter openings defined by a first distal adapter surface, wherein the first proximal adapter surface is removably connectable with the distal body surface of the fluid directing body; and
an applicator tip removably connectable with the first distal adapter surface ofthe first fluid control adapter,
wherein when the fluid directing body, the first fluid control adapter, and the applicator tip are fluidically connected, the substance applied to an application surface occurs as fluid passes from the fluid directing body, through the first fluid control adapter, and through the applicator tip depositing the substance to the application surface.

54. The modular microfluidic flow cell array of claim 53, wherein the first adapter microfluidic channel:
interacts mechanically, optically, chemically, biologically, or thermally with fluid during at least a portion of the time that fluid is contained within or passing through the first fluid control adapter;
includes mechanical architecture to introduce fluid contained within or passing through the first fluid control adapter to fluid dividing, fluid combining, fluid mixing, fluid sampling, fluid trapping, air bubble separation, or a combination thereof;
includes an electrical component to introduce fluid contained within or passing through the first fluid control adapter to electrical current, capacitance, electrophoresis, dielectrophoresis, electroshock, electrospray, electromagnetic frequency, electromagnetic field, resistance, induction, piezoelectric interaction, or a combination thereof;
a valve;
a sensor to sense a property of the fluid, wherein the sensor is an optical sensor, a chemical sensor, a microelectromechanical system sensor, an electrical sensor, a biological sensor, an NMR sensor, a dielectrophoretic sensor, a capacitance sensor, or a combination thereof;
carries a dry reagent or a fluid reagent to interact with fluid contained within or passing through the first fluid control adapter;
a thermocontroller, or
a combination thereof.

55-60. (canceled)

61. The modular microfluidic flow cell array of claim 53, further comprising a second fluid control adapter having a second adapter microfluidic channel fluidically connecting one or more proximal adapter openings defined by a second proximal adapter surface with one or more distal adapter openings defined by a second distal adapter surface, wherein the second fluid control adapter is positionable between the first fluid control adapter and the applicator tip or is interchangeable with the first fluid control adapter.

62. The modular microfluidic flow cell array of claim 61, wherein:
the second fluid control adapter is positioned between the first fluid control adapter and the applicator tip, wherein the second adapter microfluidic channel fluidically connects the first adapter microfluidic channel with an applicator flow channel of the applicator tip;
the first adapter microfluidic channel interacts mechanically, optically chemically, biologically, or thermally with fluid during at least a portion of the time that fluid is contained within or passing through the first fluid control adapter, wherein the second adapter microfluidic channel also interacts mechanically, optically, chemically, biologically, or thermally with fluid during at least a portion of the time that fluid is contained within or passing through the first fluid control adapter, and wherein the first adapter microfluidic channel and the second adapter microfluidic channel interact differently with the fluid relative to one another; or
a combinations thereof.

63. (canceled)

64. The modular microfluidic flow cell array of claim 53, wherein the applicator tip is integrated with the application surface.

65. The modular fluid control adapter system of claim 64, wherein:
a second applicator tip is also integrated with the application surface at a different location along the application surface relative to the applicator tip, wherein the second applicator tip is also removably attachable to the first distal adapter surface of the first fluid control adapter; or
the distal body surface is sealed with the first proximal adapter surface and the first distal adapter surface is sealed with a proximal tip surface of the applicator tip, and wherein the fluid directing body, the first fluid control adapter, and the applicator tip are fluidically connected together to permit fluid to pass from within the fluid directing body and through the applicator tip.

66. (canceled)

67. The modular fluid control adapter system of claim 64, wherein the first fluid control adapter;
includes flexible hollow conduit having flow channels longer than a width or a depth of the first fluid control adapter;
is configured to reserve the microfluidic connection to the applicator tip integrated with the application surface even when the fluid directing body is moved in an x-direction, a y-direction, a z-direction, or a combination of directions thereof; or
a combinations thereof.

68. (canceled)

69. The modular fluid control adapter system of claim 53, wherein:
the connection and disconnection of the fluid directing body, the first fluid control adapter, and the applicator tip occurs by automation;
the exchange of the first fluid control adapter with a second fluid control adapter occurs by automation;
adding the second fluid control adapter between the first fluid control adapter and the applicator tip occurs by automation; or
a combination thereof.

70. (canceled)

71. The modular fluid control adapter system of claim 53, wherein the connection between:
the distal body surface of the fluid directing body and the proximal adapter surface of the first fluid control adapter also makes electrical, optical, thermal, or mechanical connection;
the first distal adapter surface of the first fluid control adapter also makes electrical, optical, thermal, or mechanical connection with the applicator tip or a second fluid control adapter positioned between the first fluid control adapter and the applicator tip; or
a combinations thereof.

72. (canceled)

73. The modular fluid control adapter system of claim 53, wherein the first fluid control adapter includes multiple first adapter mnicrofluidic channels.

74. The modular fluid control adapter system of claim 53, wherein the first fluid control adapter includes one or more proximal adapter openings associated with unconnected channels that do not permit the fluid to pass into the applicator tip.

75. The modular fluid control adapter system of claim 53, wherein when the fluid directing body, the first fluid control adapter, and the applicator tip are fluidically connected and the substance has been deposited on the application surface, a return flow path is present to return the fluid through the first fluid control adapter and into the fluid directing body.

76. A method of treating an application surface for analysis of a substance, comprising:
docking a first modular microfluidic flow cell array on an application surface, wherein the first modular microfluidic flow cell array includes a fluid directing body fluidly connected to a first fluid control adapter;
flowing a first fluid through the first modular microfluidic flow cell array including through the first fluid control adapter;
removing the first fluid control adapter from the fluid directing body;
fluidly connecting a second fluid control adapter to the fluid directing body to form a second modular microfluidic flow cell array;
docking the second modular microfluidic flow cell array on the application surface; and
flowing a second fluid through the second modular microfluidic flow cell array including through the second fluid control adapter.

77. The method of claim 76, wherein the first fluid and the second fluid are the same.

78. The method of claim 76, wherein the first fluid and the second fluid are different.

79. The method of claim 76, wherein the first fluid control adapter interacts differently with the fluid compared to the second fluid control adapter.

80. The method of claim 76, wherein the first fluid control adapter, the second fluid control adapter, or both are in the form of a first applicator tip, a second applicator tip, or both, respectively.

81. The method of claim 76, wherein the first fluid control adapter, the second fluid control adapter, or both are not in the form of applicator tips, and the method further includes:
fluidly connecting a first applicator tip to the first fluid control adapter to flow the first fluid also through the first applicator tip to contact the application surface via the first applicator tip;
fluidly connecting a second applicator tip to the second fluid control adapter to flow the second fluid also through the second applicator tip to contact the application surface via the second applicator-tip: or
a combination thereof.

82. The method of claim 76, wherein flowing the first fluid, flowing the second fluid, or both includes flowing fluid for a purpose other than depositing substance spots on the application surface, but rather is for surface preparation, deposition spot washing, microfluidic channel priming, generating a reaction, removing materials, swapping materials, manipulating cells, or a combination thereof.

83. (canceled)

84. The method of claim 76, wherein flowing the first fluid results in depositing a first group of substance spots on the application surface, flowing the second fluid results in depositing a second group of substance spots on the application surface, or both.

85. The method of claim 84, further comprising depositing the first group of substance spots and the second group of substances spots at overlapping interstitial locations on the application surface.

86. The method of claim 85, wherein the overlapping interstitial locations are:
partially overlapping with one or both of the first group of substance spots or the second group of substance spots being applied to space on the application surface without the first group of substance spots; or
fully overlapping.

87. (canceled)

88. The method of claim 84, further comprising;
depositing the first group of substance spots and the second group of substances spots at non-overlapping interstitial locations on the application surface;
applying an interrogation fluid to a plurality of spots or all spots of the first group of substance spots, the second group of substances spots, or both; or
a combination thereof.

89. The method of claim 84, wherein:
unspotted areas or uniformly spotted areas of the first or second group of substance spots are used for internal referencing;
the first modular microfluidic flow cell array and the second modular microfluidic flow cell array are arranged to deposit different patterns of substance on the application surface; or
a combinations thereof.

90-91. (canceled)

92. The method of claim 91, wherein the interrogation fluid is:
applied on unspotted space on the application surface surrounding the plurality of spots or all of the spots of the first group of substance spots, the second group of substances spots, or both;
applied using an interrogation applicator tip that is fluidically connectable directly or indirectly to the fluid directing body;
fluidically connectable and disconnectable by mechanical, magnetic, or electrostatic force engagement and disengagement; or
a combination thereof.

93-94. (canceled)

95. The method of claim 84, wherein depositing the first group of substance spots, the second group of substances spots, or both includes associating location data with the first group of substance spots, the second group of substance spots, or both by engaging a location sensor identifier with the application surface, the first modular microfluidic flow cell array, the second modular microfluidic flow cell array, the first group of substance spots, the second group of substance spots, or a combination thereof.

96. The method of claim 76, wherein removing the first fluid control adapter from the fluid directing body and fluidly connecting a second fluid control adapter to the fluid directing body is carried out by;
mechanical disengagement of the fluid directing body with the first fluid control adapter and mechanical engagement of the fluid directing body with the second fluid control adapter;
magnetic disengagement of the fluid directing body with the first fluid control adapter and magnetic engagement of the fluid directing body with the second fluid control adapter;
electrostatic force disengagement of the fluid directing body with the first fluid control adapter and electrostatic force engagement of the fluid directing body with the second fluid control adapter; or
a combination thereof.

97-98. (canceled)

99. The method of claim 76, wherein removing the first applicator tip from the first fluid control adapter and fluidly connecting the second applicator tip to the second fluid control adapter is carried out by:
mechanical disengagement of the first fluid control adapter with the first applicator tip and mechanical engagement of the second fluid control adapter with the second applicator tip;
magnetic disengagement of the first fluid control adapter with the first applicator tip and magnetic engagement of the second fluid control adapter with the second applicator tip;
electrostatic force disengagement of the first fluid control adapter with the first applicator tip and electrostatic force engagement of the second fluid control adapter with the second applicator tip; or
a combination thereof.

100-101. (canceled)

102. The method of claim 76, wherein the first modular microfluidic flow cell array, the second modular microfluidic flow cell array, or both include data collection hardware or indicia to collect data related to the first fluid, the second fluid, or both; contacting the application surface, depositing substance spots on the application surface, or both.

103. The method of claim 102, wherein the data collection hardware or indicia includes an RFID tag, a 2D barcode, an electrical contact, or optics, wherein the data collection hardware is associated with a processor or processors associated with the first modular microfluidic flow cell array, the second modular mnicrofluidic flow cell array, the application surface, or a combination thereof.

104. The method of claim 102, wherein the processor or processors are configured to convey adjustments to an operation selected from changing fluid flow parameters, expanding or restricting ports, contact pressure with the application substrate, temperature adjustment, valve operation, activating sensors, electromechanical manipulations, acoustic manipulations, optical manipulations, or a combination thereof.

105. The method of claim 104, wherein the processor or processors are in the form of an internal chip or chips.

106. The method of claim 105, wherein the internal chip or chips;
are located on the first modular microfluidic flow cell array, the second modular microfluidic flow cell array, or both at a location or locations that contacts the application surface when flowing the first fluid, flowing the second fluid, or both occurs, leaving a first group of substance spots, a second group of substance spots, or both;
include an interlocking feature creating a fluid-tight seal with the application surface or withstanding pulling forces associated with release from the application surface; or
a combination thereof.

107. (canceled)

108. The method of claim 76, wherein the fluid directing body, the first fluid control adapter, the second fluid control adaptor, or a combination thereof includes chamfers or guidance indicators to enhance fluidically coupling when engaging the fluid directing body with the first fluid control adapter, the second fluid control adaptor, or both.

109. The method of claim 76, further comprising:
thermally interacting with fluid contained within or passing through the first fluid control adapter, the second fluid control adapter, or both;
mechanically interacting with fluid contained within or passing through the first fluid control adapter, the second fluid control adapter, or both by fluid dividing, fluid combining, fluid mixing, fluid sampling, fluid trapping, air bubble separation, or a combination thereof,
electrically interacting with fluid contained within or passing through the first fluid control adapter, the second fluid control adapter, or both using electrical current, capacitance, electrophoresis, dielectrophoresis, electroshock, electrospray, electromagnetic frequency, electromagnetic field, resistance, induction, piezoelectric interaction, or a combination thereof,
sensing a fluid or substance property contained within or passing through the first fluid control adapter, the second fluid control adapter, or both using an optical sensor, a chemical sensor, a microelectromechanical system sensor, an electrical sensor, a biological sensor, an NMR sensor, a dieletrophoretic sensor, a capacitance sensor, or a combination thereof,
chemically interacting with fluid contained within or passing through the first fluid control adapter, the second fluid control adapter, or both, using the first adapter microfluidic channel by contacting the fluid with a dry reagent or a fluid reagent contained therein; or
a combination thereof.

110-113. (canceled)

114. The method of claim 76, wherein removing the first fluid control adapter from the fluid directing body and fluidly connecting a second fluid control adapter to the fluid directing body occurs by automation.

115. The method of claim 76, wherein:
flow of the first fluid while the first modular microfluidic flow cell array is sealed against the application surface further includes return of the first fluid through the first fluid control adapter and into or through the fluid directing body;
flow of the second fluid while the second modular microfluidic flow cell array is sealed against the application surface further includes return of the second fluid through the second fluid control adapter and into or through the fluid directing body; or
a combination thereof.

116. The method of claim 76, further comprising undocking the first modular microfluidic flow cell array from the application surface after flowing the first fluid through the first modular microfluidic flow cell array.

117-128. (canceled)
